(19) United States
(12) Patent Application Publication
(10) Pub. No.: US 2006/0271056 A1
(43) Pub. Date: Nov. 30, 2006

(54) System and method for modular navigated osteotome

(75) Inventors: Lauralan Terrill-Grisoni, Cordova, TN (US); Patrick J. Culley, Stevenson Ranch, CA (US)

Correspondence Address: CHIEF PATENT COUNSEL;SMITH & NEPHEW, INC., 1450 BROOKS ROAD, MEMPHIS, TN, 38116, US

(73) Assignee: Smith & Nephew, Inc., Memphis, TN (US)

(21) Appl. No.: 11/431,467

(22) Filed: May 10, 2006

Related U.S. Application Data
(60) Provisional application No. 60/679,526, filed on May 10, 2005.

(51) Int. Cl. A61B 17/00

(52) U.S. Cl. 606/84

(57) ABSTRACT

An osteotome instrument for use in computer assisted surgery is disclosed. The instrument includes a shaft, a connector, a handle, and a cutter component. The handle has a proximal end portion and a distal end portion. The cutter component is connected to the handle at the distal end portion. The connector is releasably connected to the handle at the proximal end portion, and the connector is adapted to rotate about the shaft relative to the handle. A fiducial for tracking is connected to the connector.

### CROSS-REFERENCE TO RELATED APPLICATIONS

This application claims the benefit of U.S. Provisional Application No. 60/679,526, filed May 10, 2005, the disclosure of which is incorporated by reference in its entirety.

### BACKGROUND OF THE INVENTION

1. Field of the Invention

The present invention relates generally to computer assisted surgery and more particularly to instruments for computer assisted surgery.

2. Related Art

Computer-assisted surgical systems use various imaging and tracking devices and combine the image information with computer algorithms to track the position of the patient's anatomy, surgical instruments, prosthetic components, virtual surgical constructs, such as body and limb axes, and other surgical structures and components. The computer-assisted surgical systems use this data to make highly individualized recommendations on a number of parameters, including, but not limited to, patient's positioning, the most optimal surgical cuts, prosthetic component selection, and prosthetic component positioning. Orthopedic surgery, including, but not limited to, joint replacement surgery, is one of the areas where computer-assisted surgery is becoming increasingly popular.

During joint replacement surgery, diseased or damaged joints within the musculoskeletal system of a human or an animal, such as, but not limited to, a knee, a hip, a shoulder, an ankle, or an elbow joint, are partially or totally replaced with long-term surgically implantable devices, also referred to as joint implants, joint prostheses, joint prosthetic implants, joint replacements, or prosthetic joints.

Knee arthroplasty is a procedure for replacing components of a knee joint damaged by trauma or disease. During this procedure, a surgeon removes a portion of one or more knee bones forming the knee joint and installs prosthetic components to form the new joint surfaces. In the United States alone, surgeons perform approximately 250,000 total knee arthroplasties (TKAs), or total replacements of a knee joint, annually. Thus, it is highly desirable to improve this popular technique to ensure better restoration of knee joint function and shortening the patient's recovery time.

The structure of the human knee joint is detailed, for example, in “Questions and Answers About Knee Problems” (National Institute of Arthritis and Musculoskeletal and Skin Diseases (NIAMS) Information Clearinghouse National Institutes of Health (NIH), Bethesda, Md., 2001), incorporated by reference herein. The human knee joint includes essentially four bones. The lower extremity of the femur, or distal femur, attaches by ligaments and a capsule to the proximal tibia. The distal femur contains two rounded oblong eminences, the condyles, separated by an intercondylar notch. The tibia and the femur do not interlock but meet at their ends. The femoral condyles rest on the condyles of the proximal tibia. The fibula, the smaller shin bone, attaches just below the tibia and is parallel to it. The patella, or knee cap, is at the front of the knee, protecting the joint and providing extra leverage. A patellar surface is a smooth shallow articular depression between the femoral condyles at the front. Cartilage lines the surfaces of the knee bones, cushions them, and minimizes friction. Two C-shaped menisci, or meniscal cartilage, lie between the femur and the tibia, serve as pockets for the condyles, and stabilize the knee. Knee ligaments connect the knee bones and cover and stabilize the joint. The knee ligaments include the patellar ligament, the medial and lateral collateral ligaments, and the anterior cruciate ligament (ACL) and the posterior cruciate ligament (PCL). The medial collateral ligament (MCL) provides stability to the inner (medial) part of the knee. The lateral collateral ligament (LCL) provides stability to the outer (lateral) part of the knee. The anterior cruciate ligament (ACL), in the center of the knee, limits rotation and the forward movement of the tibia. The posterior cruciate ligament (PCL), also in the center of the knee, limits backward movement of the tibia. Ligaments and cartilage provide the strength needed to support the weight of the upper body and to absorb the impact of exercise and activity. Tendons, such as muscle, and cartilage are also instrumental to joint stabilization and functioning. Some examples of the tendons are popliteus tendon, which attaches popliteus muscle to the bone. Pes anserinus is the insertion of the conjoined tendons into the proximal tibia, and comprises the tendons of the sartorius, gracilis, and semitendinosus muscles. The conjoined tendon lies superficial to the tibial insertion of the MCL. The iliotibial band extends from the thigh down over the knee and attaches to the tibia. In knee flexion and extension, the iliotibial band slides over the lateral femoral epicondyle. The knee capsule surrounds the knee joint and contains lubricating fluid synovium.

A healthy knee allows the leg to move freely within its range of motion while supporting the upper body and absorbing the impact of its weight during motion. The knee has generally six degrees of motion during dynamic activities: three rotations (flexion/extension angulations, axial rotation along the long axis of a large tubular bone, also referred to as interior/exterior rotation, and varus/valgus angulations); and three translations (anterior/posterior, medial/lateral, and superior/inferior).

A total knee arthroplasty, or TKA, replaces both the distal femur and the proximal tibia of the damaged or diseased knee with artificial components made of various materials, including, but not limited to, metals, ceramics, plastics, or their combinations. These prosthetic knee components are attached to the bones, and the existing soft tissues are used to stabilize the artificial knee. During TKA, after preparing and anesthetizing the patient, the surgeon makes a long incision along the front of the knee and positions the patella to expose the joint. After exposing the ends of the bones, the surgeon removes the damaged tissue and cuts, or resects, the portions of the tibial and femoral bones to prepare the surfaces for installation of the prosthetic components.

To properly prepare femoral surfaces to accept the femoral and tibial components of the prosthetic knee, the surgeon needs to accurately determine the position of and perform multiple cuts. The surgeon may use various measuring and indexing devices to determine the location of the cut, and various guiding devices, such as, but not limited to, guides, jigs, blocks and templates, to guide the saw blades to accurately resect the bones. After determining the desired position of the cut, the surgeon usually attaches the guiding device to the bone using appropriate fastening mechanisms, including, but not limited to, pins and screws. Attachment to structures already stabilized relative to the bone, such as intramedullary rods, can also be employed. After stabilizing the guiding device at the bone, the surgeon uses the guiding component of the device to direct the saw blade in the plane of the cut.

After preparation of the bones, the knee is tested with the trial components. Soft-tissue balancing, including any necessary surgical release or contraction of the knee ligaments and other soft tissues, is performed to ensure proper post-operative functioning of the knee. Both anatomic (bone-derived landmarks) and dynamic or kinematic (ligament and bone interactions during the knee movement) data may be considered when determining surgical cuts and positioning of the prosthetic components. After ligament balancing and proper selection of the components, the surgeon installs and secures the tibial and femoral components. The patella is typically resurfaced after installation of the tibial and femoral component, and a small plastic piece is often placed on the rear side, where it will cover the new joint. After installation of the knee prosthesis, the knee is closed according to conventional surgical procedures. Post-operative rehabilitation starts shortly after the surgery to restore the knee's function.

In order to ensure proper post-operative functioning of the prosthetic knee after total knee replacement (TKR) surgery, a surgeon must properly position and align the prosthetic knee components and properly balance the knee ligaments, including any necessary surgical release or contraction. Improper positioning and misalignment of the prosthetic knee components and improper ligament balancing commonly cause prosthetic knees to fail, leading to revision surgeries. This failure increases the risks associated with knee replacement, especially because many patients requiring prosthetic knee components are elderly and highly prone to the medical complications resulting from multiple surgeries. Also, having to perform revision surgeries greatly increases the medical costs associated with the restoration of the knee function. In order to prevent premature, excessive, or uneven wear of the artificial knee, the surgeon must implant the prosthetic device so that its multiple components articulate at exact angles, are properly supported, and are stabilized by accurately balanced knee ligaments. Thus, correctly preparing the bone for installation of the prosthetic components by precisely determining and accurately performing all the required bone cuts and correct ligament balancing are vital to the success of TKR.

Traditionally, the surgeons rely heavily on their experience to determine where the bone should be cut, to select, align and place the knee prosthetic components, and to judge how the knee ligaments should be contracted or released to ensure proper ligament balancing. With the advent of computer-assisted surgery, surgeons started using computer predictions in determining surgical cutting planes, ligament balancing, and selection, alignment and positioning of the prosthetic components. In the conventional TKR methods, anatomical (bone-derived landmarks) and dynamic or kinematic (ligament and bone interactions during the knee movement) data are usually considered separately when determining surgical cuts and positioning of the components of the prosthetic knee. Generally, conventional methods are either excessively weighted toward anatomical landmarks on the leg bones or soft tissue balancing (such as adjustment of lengths and tensions of the knee ligaments). Often, only femoral landmarks are considered when determining femoral component positioning and only tibial landmarks are considered when determining tibial component positioning. In the conventional techniques, irreversible bone cuts in the knee are usually made prior to considering the dynamic balance of the surrounding soft tissue envelope.

One conventional method of determining the femoral resection depth is anterior referencing, which is primarily focused on placing the femoral component in a position that does not notch or stuff anteriorly. The method largely ignores the kinematics of the tibio-femoral joint. Another conventional method, posterior referencing of the femoral resection depth, uses the posterior femoral condyles as a reference for resection but ignores the dynamic tissue envelope. As an additional drawback, varus and valgus knee deformities affect the resection depth determination by anterior and posterior referencing.

Determining the resection depth based on the surrounding soft tissue envelope is also problematic. If the resection determination is made before the envelope is adequately released, the resection may be inappropriately placed and, likely, excessive. Generally, ignoring the important anatomical landmarks can result in significant malrotation of the femoral component with respect to the bony anatomy.

Conventional anatomical methods of determining femoral component positioning employ the anatomical landmarks, such as epicondylar axes, Whiteside's line, and the posterior condyles. By using these anatomical landmarks and ignoring the state of the soft tissue envelope around the knee, the methods encounter certain limitations. For example, the epicondylar axes rely on amorphous knee structures and, thus, are not precisely reproducible. Typically, several sequential determinations of the epicondylar axis produce differing results. Exposing the condyles to determine the epicondylar axis requires significant tissue resection and increases risks to the patient and healing time. Whiteside's line is based on the orientation of the trochlea and is also not precisely reproducible. Furthermore, the line is not correlated with the bony anatomy and ligaments of the tibio-femoral joint in either flexion or extension.

While easily reproduced, resection of the femur parallel to the posterior femoral condyles is potentially inaccurate because it ignores the dynamic status of the surrounding soft tissue envelope. Further, the deformity and wear pattern of the arthritic knee is incorporated into the decision. For example, varus knees typically have significant cartilage wear in the posterior portion of the medial femoral condyle, while the lateral femoral condyle often has a normal cartilage thickness posteriorly. This results in excessive rotation of the femoral component upon placement. Knees with valgus malalignment and lateral compartment arthrosis typically have full-thickness cartilage loss in the lateral femoral condyle and under-development, or hypoplasia, of the condyle. The use of posterior referencing to determine femoral component rotation typically results in excessive internal rotation of the femoral component.

Determining femoral component rotation based on the surrounding soft tissue envelope is attractive because resection of the femur perpendicular to the tibia at 90 degrees of flexion with the ligaments under distraction assures a rectangular flexion gap. However, this method ignores the anatomy of the femur and the extent of the ligament release. For example, if the knee is severely varus and is inadequately released, then the medial side will remain too tight, which results in excessive external rotation of the femoral component. The opposite problem arises due to inadequate released knees with valgus-flexion contractures.

U.S. Patent Application Publication No. 2003/0153978 A1, published on Aug. 14, 2003, having an Application No. of Ser. No. 10/072,372, and listing Leo A. Whiteside as the sole inventor, incorporated by reference herein, discloses a system, apparatus, and method for soft tissue balancing. The computer-assisted surgery system compares the kinematics of the trial prosthetic joint components installed in a knee joint with the kinematics of the normal joint and provides the surgeon with the information allowing the balancing of the ligaments of the installed prosthetic joint. A video camera registers, and a computer determines, the position and orientation of the trial components with respect to each other and the kinematics of the trial components relative to one another, identifies anomalies between the observed kinematics of the trial components and the known kinematics in a normal knee, and then suggests to the surgeon which of the ligaments should be adjusted to achieve a balanced knee. Essentially, the femur and the tibia are cut first, and the knee kinematics are checked after the irreversible bone cuts are made and trial prosthetic components are installed. The method is not suitable for prediction of the optimal bone cuts based on the combination of the anatomic and the kinematic data and does not employ the combination of such data in prosthetic component positioning and ligament balancing. Furthermore, the method requires the use of the video camera to acquire the images of the installed trial components and employs complex “machine vision” algorithm to deduce the position of the prosthetic components and other landmarks from the images.

Another method of computer assisted ligament balancing provides for ligament balancing prior to femoral resection and prosthetic component positioning but relies on using a tensor that is inserted between the femur and the tibia and separates the ends of the tibia and the femur during kinematic testing. The method relies extensively on visual images and surgeon judgment in ligament alignment, selection of the implant geometry and size, determination of the femoral resection plane, and prosthetic component positioning.

There is an unrealized need for improved systems and methods for computer-assisted soft-tissue balancing, component placement, and surgical resection planning during TKA. Particularly, the field of computer assisted TKA needs improved methods and systems that consider and correlate both anatomical landmarks and dynamic interactions of the knee bones and soft tissues. Systems and methods are also desired that incorporate soft tissue balancing and component placement algorithms for quantitative assessment of the anatomical and dynamic aspects of the human knee and provide recommendations on soft tissue balancing, component selection and/or placement, and propose bone resection planes based on iterative convergence of the anatomical and the dynamical factors. Preferably, the desired systems and methods comprise a logic matrix for quantitative assessment of the state of the knee's soft tissues. Systems and methods are also needed that allow for prosthetic component selection and/or placement, soft tissue balancing, and resection planning in a variety of combinations and sequences, based on the patient's need and the surgeon's preference. There is also a need in the systems and methods that allow for component selection and/or placement, soft tissue balancing, and resection planning prior to making any surgical cuts.

In unicompartmental arthritis of the knee, high tibial osteotomy (“HTO”) is a treatment of choice. HTO is a common treatment for tibia vara (bow legs). An osteotomy is a surgical procedure to realign a bone in order to change the biomechanics of a joint, especially to change the force transmission through a joint. HTO is a corrective surgical procedure in which the upper part of the tibia is resected so that the lower limb can be realigned. The purpose of HTO is to realign the deformed tibial plateau to shift the load bearing into the unaffected compartment of the knee.

There are three types of HTO: closing wedge, open wedge, and cylindrical barrel. The closing wedge HTO is the most common procedure, and it involves realignment of the bone by removal of a lateral wedge of bone from the proximal tibia. The wedge is first planned on a frontal-plane standing X-ray by drawing a wedge of the desired correction angle, where the wedge's upper plane is parallel to the tibial plateau and the lower plane is above the tibial tubercle. Ideally, the wedge will produce a hinge of cortical bone approximately 2-5 mm in thickness.

Upon surgical exposure of the proximal tibia, the correction is mapped to the bones of the patient with a ruler or a jig system. The surgery is then performed either free-hand or with the assistance of Kirschner wires (K-wires) as cutting guides. Intraoperative fluroscopic X-ray is often employed for verification before and during the procedure.

Unlike total knee arthroplasty (“TKA”), HTO preserves the joint's original cartilaginous surfaces and corrects the fundamental mechanical problem of the knee. This advantage is especially important to young active patients because TKA has a greater probability of earlier failure in active patients.

However, problems remain in HTO performance. A major difficulty with HTO is that the outcome is sometimes not acceptably predictable because it is difficult for a surgeon to attain the desired correction angle. Current instrumentation cannot accurately produce the desired resection from preoperative plans. On average, the margin of error is reported between 6 and 14 degrees. Technical difficulties also arise from the use of fluoroscopy, such as image-intensifier nonlinearities and distortions that compromise accuracy and parallax errors that can provide misleading angular and positional guidance. Additionally, the use of continual fluoroscopic imaging is sometimes required, thus exposing the surgeon and assistants to radiation.

Several providers have developed and marketed improved cutting jigs that have improved the accuracy of the resection in HTO. However, extensive fluoroscopic time is still needed for the positioning of the jigs. Inaccurate pin placement can also affect the accuracy of the alignment of the resection, thus increasing shear stresses across the osteotomy. Other providers have developed various forms of imaging systems for use in surgery. Many are based on computed tomography (CT) scans and/or magnetic resonance imaging (MRI) data or on digitized points on the anatomy. Other systems align preoperative CT scans, MRIs or other images with intraoperative patient positions. A preoperative planning system allows the surgeon to select reference points and to determine the final implant position. Intraoperatively, the system calibrates the patient position to that preoperative plan, such as using a “point cloud” technique, and can use a robot to make femoral and tibial preparations.

In general, there is a need for systems and methods that are flexible and allow the surgeon to operate in accordance with the patient's need and the surgeon's own preferences and experience, that do not limit the surgeon to a particular surgical technique or method, and that allow for easy adaptation of the existing surgical techniques and method to computer-assisted surgery, as well as for the improvement of and development of new surgical techniques and methods. The field of computer-assisted surgery is in need of the improved systems and methods for computer-assisted soft-tissue balancing, component placement, and surgical resection planning during TKA that are versatile, provide reliable recommendations to the surgeon, and result in improved restoration of the knee function and patient's recovery as compared to the conventional methods. Further, there is a continuing need for an intraoperative planning system and process for performing HTO's with minimal fluroscopic exposure. There is also a need for a system and process that allows improved accuracy in performing the wedge resection and in placing pins or staples. Some or all, or combinations of some, of these needs are met in various systems and processes according to various embodiments of the invention.

### SUMMARY OF THE INVENTION

It is in view of the above problems that the present invention was developed. The invention is an osteotome instrument for use in computer assisted surgery. The instrument includes a shaft, an impact member, a connector, an array, a handle, and a cutter component. The shaft has a first end portion and a second end portion, and the impact member is connected to the shaft at the first end portion. The connector is slidably connected to the shaft, and the array is connected to the connector. The handle is juxtaposed or adjacent to the connector and mounted about the shaft. The handle has a proximal portion and a distal portion, and the cutter component is connected to the handle at the distal portion.

The modular navigated osteotome is an orthopaedic instrument that is used in conjunction with a computer aided surgery system. As an example, it may be used to release soft tissues and/or resect specific soft tissues and bony anatomy in the body. The instrument contains navigation paraphernalia, such as optical trackers, electromagnetic fiducials, ultrasonic arrays, radiofrequency identification devices, etc., by which the navigation computer can locate the instrument relative to the operative anatomy. Navigation paraphernalia are also attached to the body in the standard fashion so that the navigation computer can locate the instrument relative to the anatomy.

The anatomy may be landmarked through imageless modalities, such as point selection, surface selection, etc., or, optionally, the anatomy may be landmarked via conventional imaging modalities, such as fluoroscopy, computed tomography (CT), magnetic resonance imaging (MRI), ultrasonic imaging, so that the location of the tissue to be resected can be located in three-dimensional space by the computer and consequently the navigated osteotome.

The instrument possesses a handle that contains at least some navigation paraphernalia. The instrument also possesses a cutting device that is reasonably secured to the instrument. In this fashion, the cutting device can be changed to different configurations to accommodate different anatomical structures and locations in the body during the same procedure or can be replaced when broken or dull. The cutting device may or may not contain navigation paraphernalia.

The cutting device orientation can be known by fastening the cutting device to the instrument shaft in such a way that the cutting device always has the same orientation. For example, the instrument shaft may have a keyway. Alternatively, the cutting device can be releasably secured to the instrument so that the orientation can be changed but is still known to the computer.

The navigated instrument utilizes an array mount component and the array may be adjustable about the shaft for optimum surgeon comfort and camera visibility. For this purpose, the mechanism is attached or connected to the instrument handle of the osteotome.

The array, the rotation mechanism, the instrument handle, and the cutting device may be modular such that one or more of the components can be discarded or refurbished. For example, the cutting device may be discarded if it becomes dull or bent. The modularity makes the device more economical. It also opens up the opportunity to create additional sizes of the osteotomes and different tip configurations.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 is a schematic view of a computer assisted surgery system.

FIG. 2 is a view of a knee prepared for surgery to which fiducials have been attached.

FIG. 3 is a view of a portion of a leg prepared for surgery with a C-arm for obtaining fluoroscopic images associated with a fiducial.

FIG. 4 is a fluoroscopic image of free space rendered on a monitor.

FIG. 5 is a fluoroscopic image of femoral head rendered on a monitor.

FIG. 6 is a fluoroscopic image of a knee rendered on a monitor.

FIG. 7 is a fluoroscopic image of a tibia distal end rendered on a monitor.

FIG. 8 is a fluoroscopic image of a lateral view of a knee rendered on a monitor.

FIG. 9 is a fluoroscopic image of a lateral view of a knee.

FIG. 10 is a fluoroscopic image of a lateral view of a tibia distal end.

FIG. 11 illustrates a probe being used to register a surgically related component for tracking.

FIG. 12 illustrates a probe being used to designate landmarks on bone structure for tracking.

FIG. 13 is a screen face produced during designation of landmarks to determine a femoral mechanical axis.

FIG. 14 is a screen face produced during designation of landmarks to determine an epicondylar axis.

FIG. 15 is a screen face produced during designation of landmarks to determine an anterior-posterior axis.

FIG. 16 is a screen face showing mechanical and other axes which have been established.

FIG. 17 is another screen face showing mechanical and other axes which have been established.

FIG. 18 illustrates a pivot pin being placed in the tibia.

FIG. 19 illustrates tibial cutting jigs.

FIG. 20 illustrates proximal and distal cutting jigs being placed on the tibia around the pivot pin.

FIG. 21 illustrates a first embodiment of an osteotome instrument.

FIG. 22 illustrates a second embodiment of the osteotome instrument.

FIG. 23 illustrates in a side view one embodiment of a connector.

FIG. 24 illustrates a front view of the connector shown in FIG. 23.

FIG. 25 illustrates in an end view one embodiment of a handle.

FIG. 26 illustrates in a side view the handle shown in FIG. 25.

FIG. 27 illustrates a first embodiment of a receiver.

FIG. 28 illustrates a first embodiment of a projection.

FIG. 29 illustrates a second embodiment of the receiver.

FIG. 30 illustrates a second embodiment of the projection.

FIG. 31 illustrates a first embodiment of a tip portion.

FIG. 32 illustrates a second embodiment of a tip portion.

FIG. 33 illustrates a third embodiment of a tip portion.

FIG. 34 illustrates a fourth embodiment of a tip portion.

FIG. 35 illustrates a fifth embodiment of a tip portion.

FIG. 36 illustrates a first embodiment of a calibration unit.

FIG. 37 illustrates an array having a divot.

FIG. 38 illustrates in a perspective view a second embodiment of a calibration unit.

FIG. 39 illustrates in a front view the calibration unit shown in FIG. 38.

FIG. 40 illustrates in a sectional side view the calibration unit shown in FIG. 39.

FIG. 41 is a screen face produced which assists in navigation and/or placement of a distal cutting jig.

FIG. 42 illustrates a tibia that has been stapled after a closed wedge resection.

FIGS. 43-52 illustrate use of the osteotome instrument in soft tissue balancing.

### DETAILED DESCRIPTION OF THE PREFERRED EMBODIMENTS

The following description of the preferred embodiment(s) is merely exemplary in nature and is in no way intended to limit the invention, its application, or uses.

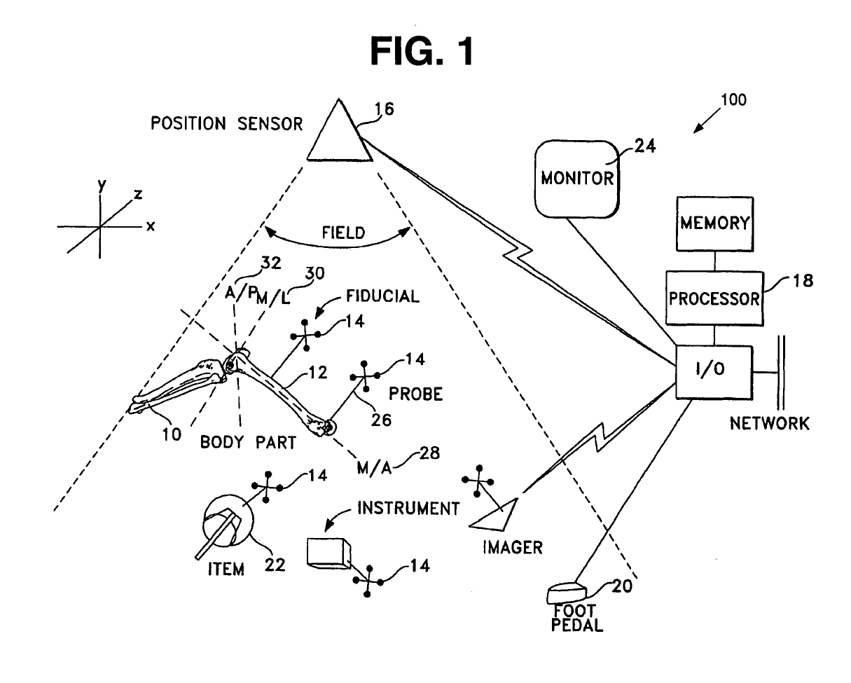

FIG. 1 is a schematic view showing one embodiment of a computer assisted surgery system 100. The computer assisted surgery system 100 uses computer capacity, including standalone and/or networked, to store data regarding spatial aspects of surgically related items and virtual constructs or references including body parts, implements, instrumentation, trial components, prosthetic components and rotational axes of body parts. Any or all of these may be physically or virtually connected to or incorporate any desired form of mark, structure, component, or other fiducial or reference device or technique which allows position and/or orientation of the item to which it is attached to be sensed and tracked, preferably in three dimensions of translation and three degrees of rotation as well as in time if desired. In some embodiments, orientation of the elements on a particular fiducial varies from one fiducial to the next so that sensors may distinguish between various components to which the fiducials are attached in order to correlate for display and other purposes data files or images of the components. In the embodiment depicted in FIG. 1, “fidicuals” are reference frames each containing at least three, sometimes more, reflective elements, such as spheres reflective of lightwave or infrared energy, or active elements, such as light emitting diodes (LEDs). In certain embodiments, some fiducials use reflective elements and some use active elements, both of which may be tracked by preferably two, sometimes more infrared sensors whose output may be processed in concert to geometrically calculate position and orientation of the item to which the fiducial is attached.

Position/orientation tracking sensors and fiducials need not be confined to the infrared spectrum. Any electromagnetic, electrostatic, light, sound, radiofrequency or other desired technique may be used. Alternatively, each item, such as a surgical implement, instrumentation component, trial component, implant component or other device, may contain its own “active” fiducial, such as a microchip with appropriate field sensing or position/orientation sensing functionality and communications link, such as spread spectrum Radio Frequency link, in order to report position and orientation of the item. Such active fiducials, or hybrid active/passive fiducials, such as transponders, can be implanted in the body parts or in any of the surgically related devices mentioned above, or conveniently located at their surface or otherwise as desired. Fiducials may also take the form of conventional structures, such as a screw driven into a bone, or any other three-dimensional item attached to another item. What is significant is that the position and orientation of the three-dimensional item can be tracked in order to track the position and orientation of body parts and/or surgically related items. Hybrid fiducials may be partly passive and partly active such as inductive components or transponders that respond with a certain signal or data set when queried by sensors.

The system 100 employs a computer to calculate and store reference axes of body components. In a High Tibial Osteotomy (HTO) for example, the mechanical axis of the femur and/or tibia may be stored. From these stored axes, the system 100 tracks the position of the instrumentation and osteotomy guides so that bone resections are optimally located. Furthermore, during trial reduction of the knee, the system 100 provides feedback on the balancing of the ligaments in a range of motion and under varus/valgus, anterior/posterior and rotary stresses and can suggest or at least provide more accurate information than in the past about which ligaments the surgeon should release in order to obtain correct balancing, alignment and stability. The system 100 can also suggest modifications to implant size, positioning, and other techniques to achieve optimal kinematics. The system 100 can also include databases of information regarding tasks, such as ligament balancing, in order to provide suggestions to the surgeon based on performance of test results as automatically calculated by the computer.

FIG. 1 is a schematic view showing one embodiment of the system 100 and one version of a setting in which surgery on a knee, in this case a High Tibial Osteotomy, may be performed. The system 100 can track various body parts, such as tibia 12 and femur 10, to which fiducials of the sort described above or any other sort may be implanted, attached, or otherwise associated physically, virtually, or otherwise. In the embodiment shown in FIG. 1, fiducials 14 are structural frames some of which contain reflective elements, some of which contain LED active elements, some of which can contain both, for tracking using stereoscopic infrared sensors suitable, at least operating in concert, for sensing, storing, processing and/or outputting data (“tracking”) relating to position and orientation of fiducials 14 and thus components, such as tibia 12 and femur 10, to which they are attached or otherwise associated. Position/orientation sensor 16 may be any sort of sensor functionality for sensing position and orientation of fiducials 14 and therefore items with which they are associated, according to whatever desired electrical, magnetic, electromagnetic, sound, physical, radio frequency, or other active or passive technique. In the embodiment depicted in FIG. 1, position sensor 16 is a pair of infrared sensors disposed on the order of a meter, sometimes more, sometimes less, apart and whose output can be processed in concert to provide position and orientation information regarding fiducials 14.

In the embodiment shown in FIG. 1, computing functionality 18 can include processing functionality, memory functionality, input/output functionality whether on a standalone or distributed basis, via any desired standard, architecture, interface and/or network topology. Computing functionality 18 may be connected to a screen or monitor 24 on which graphics and data may be presented to the surgeon during surgery. In some embodiments, the monitor 24 has a tactile interface so that the surgeon can point and click on screen for tactile screen input in addition to or instead of, if desired, keyboard and mouse conventional interfaces. Additionally, a foot pedal 20 or other convenient interface may be coupled to computer functionality 18 as can any other wireless or wireline interface to allow the surgeon, nurse or other desired user to control or direct computer functionality 18 in order to, among other things, capture position/orientation information when certain components are oriented or aligned properly. Items 22, such as trial components, instrumentation components, or implants, may be tracked in position and orientation relative to body parts, such as tibia 12 and femur 10, using fiducials 14.

Computing functionality 18 can process, store and output on monitor 24 position and orientation information and other various forms of data that correspond in whole or part to body parts, such as tibia 10 and femur 12, and other components, such as item 22. For example, in the embodiment shown in FIG. 1, tibia 10 and femur 12 are shown in cross-section or at least various internal aspects of them, such as bone canals and surface structure, are shown using fluoroscopic images. These images may be obtained using a C-arm attached to a fiducial 14. The body parts, for example, tibia 12 and femur 10, also have fiducials attached. When the fluoroscopy images are obtained using the C-arm with fiducial 14, a position/orientation sensor 16 “sees” and tracks the position of the fluoroscopy head as well as the positions and orientations of the tibia 12 and femur 10. The computer 18 stores the fluoroscopic images with this position/orientation information, thus correlating position and orientation of the fluoroscopic image relative to the relevant body part or parts. Thus, when the tibia 12 and corresponding fiducial 14 move, the computer automatically and correspondingly senses the new position of tibia 12 in space and can correspondingly move implements, instruments, references, trials and/or implants on the monitor 24 relative to the image of tibia 12. Similarly, the image of the body part can be moved, both the body part and such items may be moved, or the on-screen image may otherwise be presented to suit the preferences of the surgeon or others and carry out the imaging that is desired. Similarly, when an item 22, such as a pivot pin, that is being tracked moves, its image moves on monitor 24 so that the monitor shows the item 22 in proper position and orientation on monitor 24 relative to the femur 10. The pin 22 can thus appear on the monitor 24 in proper or improper alignment with respect to the mechanical axis and other features of the femur 10, as if the surgeon were able to see into the body in order to navigate and position the pin 22 properly.

The computer functionality 18 can also store data relating to configuration, size and other properties of items 22, such as implements, instrumentation, trial components, implant components and other items used in surgery. When those are introduced into the field of position/orientation sensor 16, computer functionality 18 can generate and display overlain or in combination with the fluoroscopic images of the body parts, computer generated images of implements, instrumentation components, trial components, implant components and other items 22 for navigation, positioning, assessment and other uses.

Additionally, computer functionality 18 can track any point in the field of position/orientation sensor 16 by using a designator or a probe 26. The probe 26 also can contain or be attached to a fiducial 14. The surgeon, nurse, or other user touches the tip of probe 26 to a point, such as a landmark on bone structure, and actuates the foot pedal 20 or otherwise instructs the computer 18 to note the landmark position. The position/orientation sensor 16 “sees” the position and orientation of fiducial 14 and “knows” where the tip of probe 26 is relative to that fiducial 14. Thereafter, computer functionality 18 calculates, stores, and may display on monitor 24 whenever desired and in whatever form or fashion or color, the point or other position designated by probe 26 when the foot pedal 20 is hit or other command is given. Thus, probe 26 can be used to designate landmarks on bone structure in order to allow the computer 18 to store and track, relative to movement of the bone fiducial 14, virtual or logical information, such as mechanical axis 28, medial laterial axis 30 and anterior/posterior axis 32 of femur 10, tibia 12 and other body parts in addition to any other virtual or actual construct or reference.

Optionally, the system 100 may incorporate systems and process that capture and correlate fluoroscopic images with body parts and related constructs. For example, the system 100 may incorporate the so-called FluoroNAV system and software provided by Medtronic Sofamor Danek Technologies. Such systems or aspects of them are disclosed in U.S. Pat. Nos. 5,383,454; 5,871,445; 6,146,390; 6,165,81; 6,235,038 and 6,236,875, and related (under 35 U.S.C. Section 119 and/or 120) patents, which are all incorporated herein by this reference. Any other desired systems can be used as mentioned above for imaging, storage of data, tracking of body parts and items and for other purposes.

The FluoroNav system requires the use of reference frame-type fiducials 14 which have four, and in some cases five, elements tracked by infrared sensors for position/orientation of the fiducials and thus of the body part or item 22. As examples, implements, instrumentation, trial components, implant components, other devices or structure may be tracked using frame-type fiducials 14. Such systems also may use the probe 26 which the surgeon can use to select, designate, register, or otherwise make known to the system a point or points on the anatomy or other locations by placing the probe as appropriate and signaling or commanding the computer to note the location of, for instance, the tip of the probe 26. The FluoroNav system also tracks position and orientation of a C-arm used to obtain fluoroscopic images of body parts to which fiducials have been attached for capturing and storage of fluoroscopic images keyed to position/orientation information as tracked by the sensors 16. Thus, the monitor 24 can render fluoroscopic images of bones in combination with computer generated images of virtual constructs and references together with implements, instrumentation components, trial components, implant components and other items used in connection with surgery for navigation, resection of bone, assessment and other purposes.

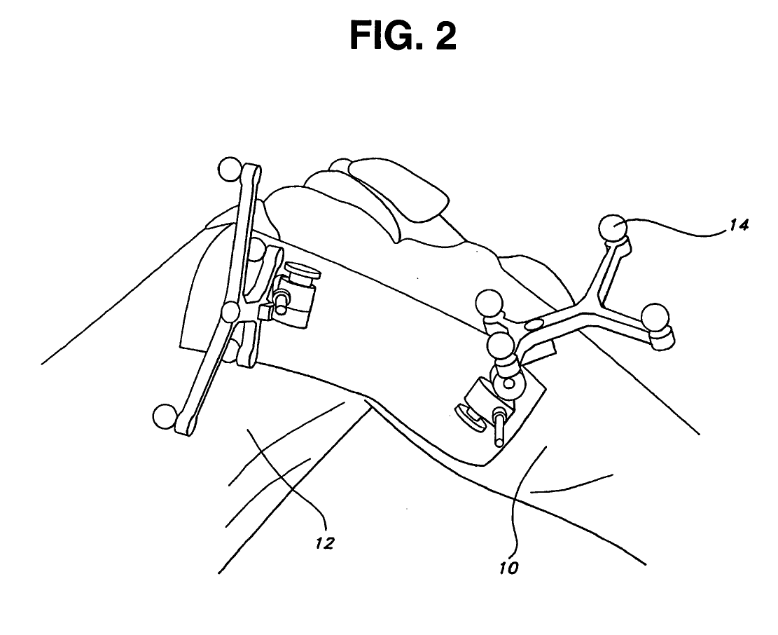

FIG. 2 shows a human knee in the surgical field, as well as the corresponding femur and tibia, to which fiducials 14 have been rigidly attached. Attachment of fiducials 14 may be accomplished using structure that withstands vibration of surgical saws and other phenomenon that occur during surgery without allowing any substantial movement of fiducial 14 relative to body part being tracked by the system.

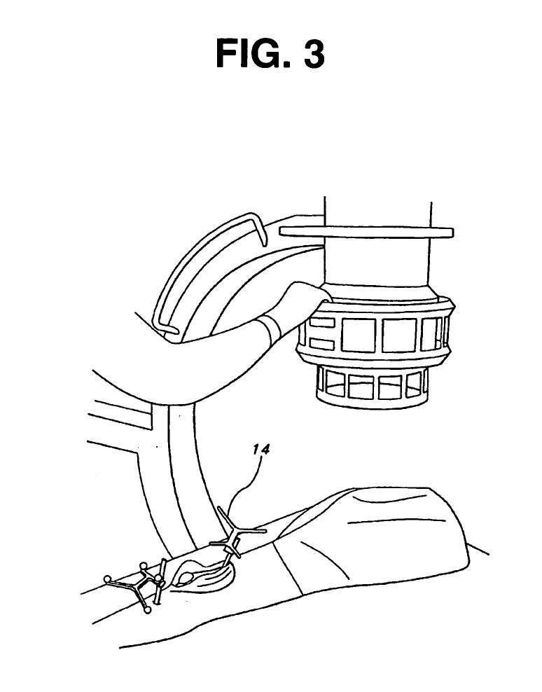

FIG. 3 shows fluoroscopy images being obtained of the body parts with fiducials 14 attached. The fiducial 14 on the fluoroscopy head in this embodiment is a cylindrically shaped cage which contains LEDs or “active” emitters for tracking by the sensors 16. Fiducials 14 attached to tibia 12 and femur 10 can also be seen. The fiducial 14 attached to the femur 10 uses LEDs instead of reflective spheres and is active, fed power by the wire seen extending into the bottom of the image.

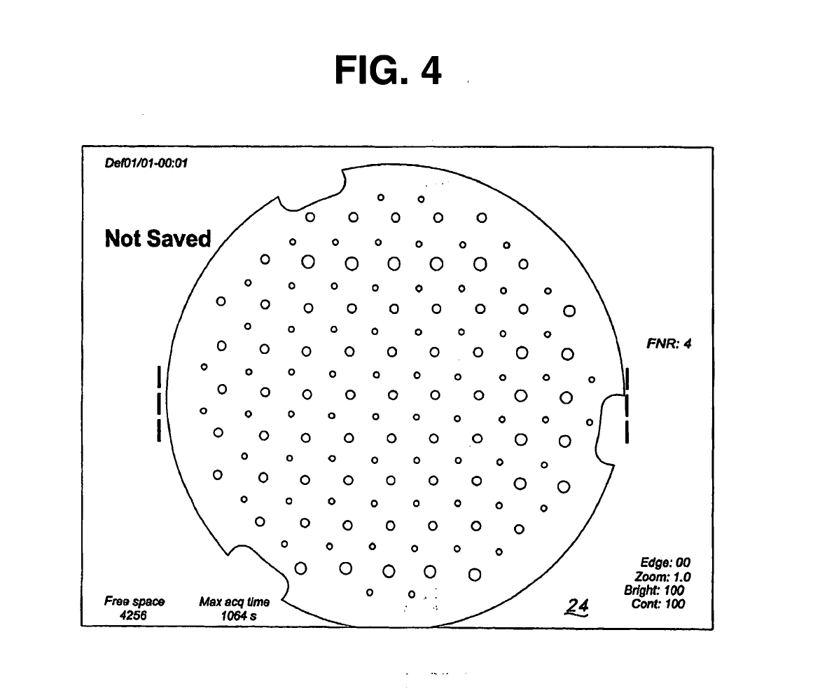

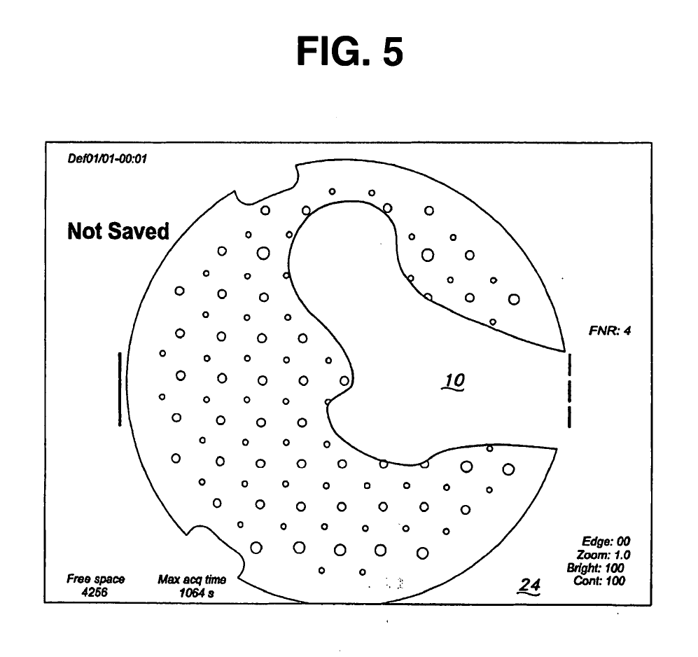

FIGS. 4-10 are fluoroscopic images shown on monitor 24 obtained with position and/or orientation information received by, noted and stored within computer 18. FIG. 4 is an open field with no body part image but which shows the optical indicia that may be used to normalize the image obtained using a spherical fluoroscopy wave front with the substantially flat surface of the monitor 24. FIG. 5 shows an image of the femur 10 head. This image is taken in order to allow the surgeon to designate the center of rotation of the femoral head for purposes of establishing the mechanical axis and other relevant constructs relating to of the femur according to which the wedge of bone will ultimately be resected. Such center of rotation can be established by articulating the femur within the acetabulum or a prosthesis to capture a number of samples of position and orientation information and in turn to allow the computer to calculate the average center of rotation. A surgeon may use the probe 26 to designate a number of points on the femoral head and allow the computer to calculate the geometrical center or a center that corresponds to the geometry of points collected. Additionally, graphical representations, such as controllably sized circles displayed on the monitor, can be fitted by the surgeon to the shape of the femoral head on planar images using tactile input on screen to designate the centers according to that graphic, such as are represented by the computer as intersection of axes of the circles. Other techniques for determining, calculating or establishing points or constructs in space, whether or not corresponding to bone structure, may also be used.

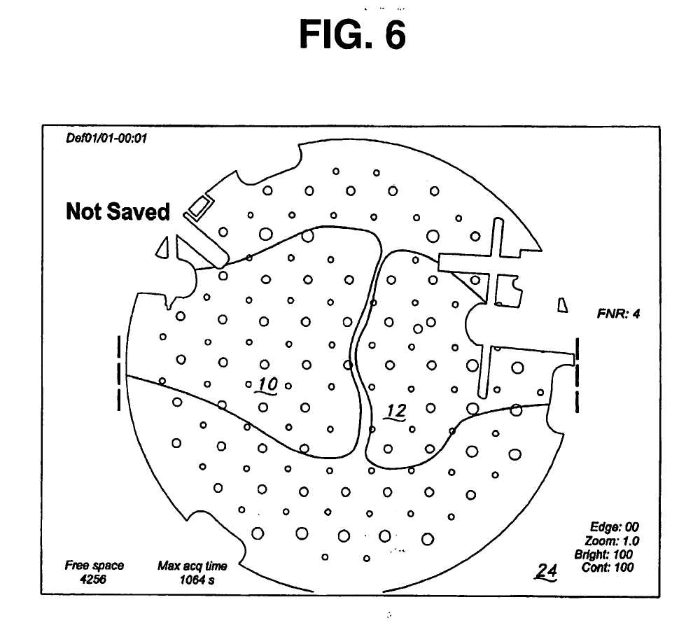

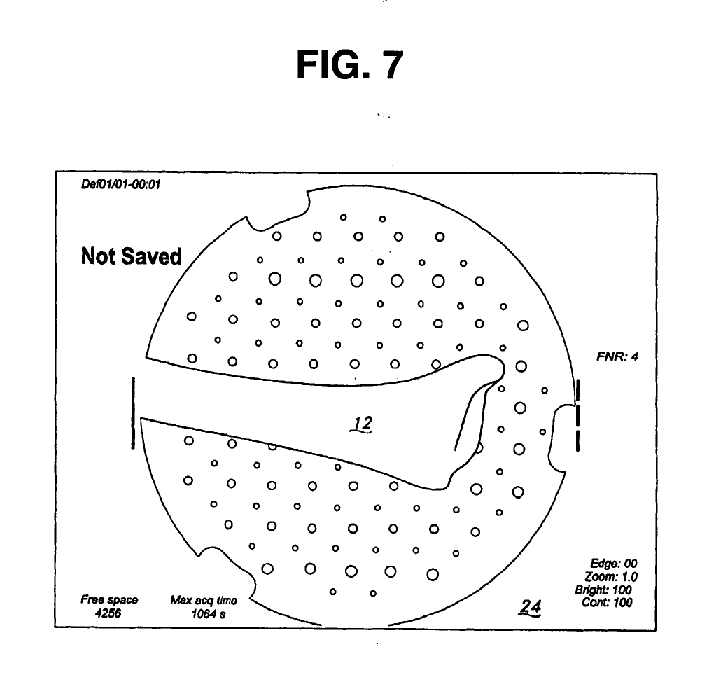

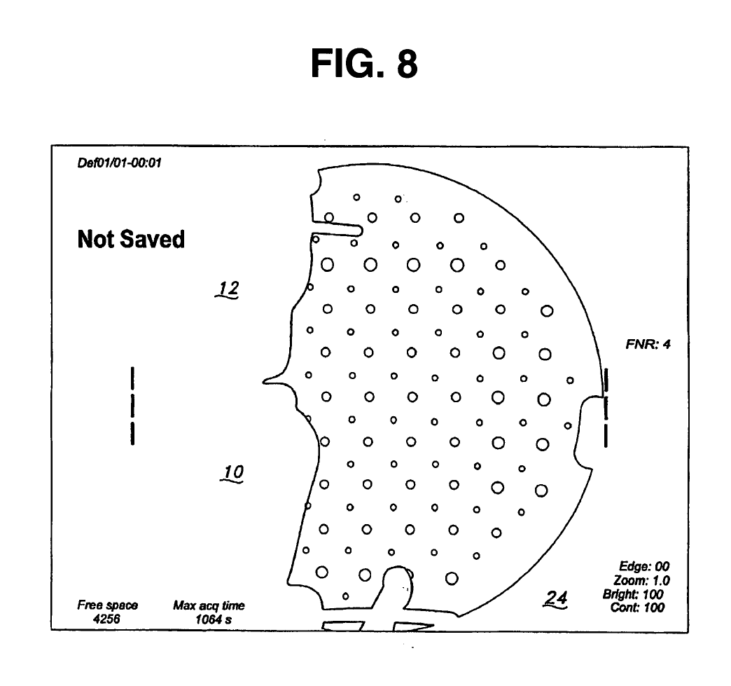

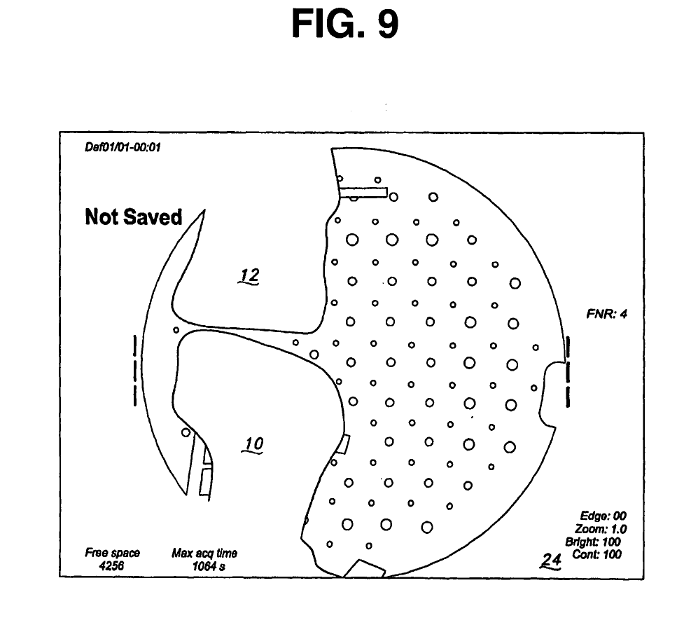

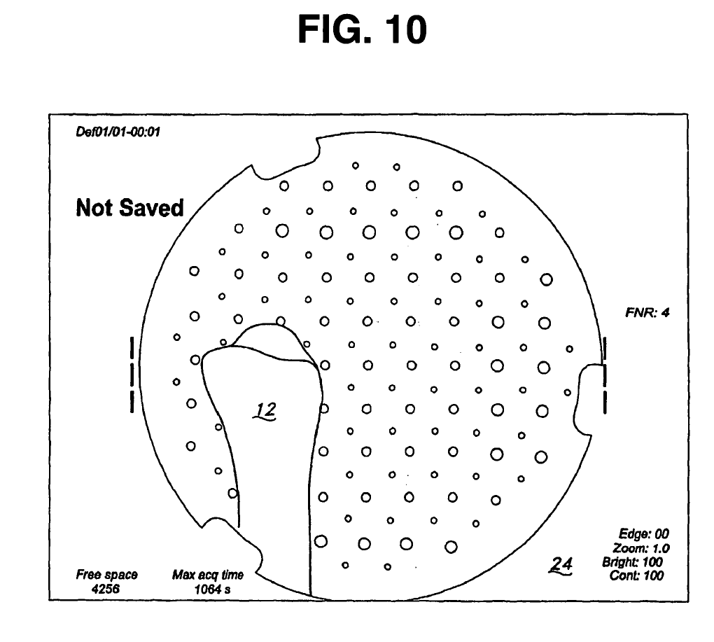

FIG. 5 shows a fluoroscopic image of the femoral head, while FIG. 6 shows an anterior/posterior view of the knee that can be used to designate landmarks and establish axes or constructs such as the mechanical axis or other rotational axes. FIG. 7 shows the distal end of the tibia, and FIG. 8 shows a lateral view of the knee. FIG. 9 shows another lateral view of the knee, while FIG. 10 shows a lateral view of the distal end of the tibia.

### Registration of Surgically Related Items

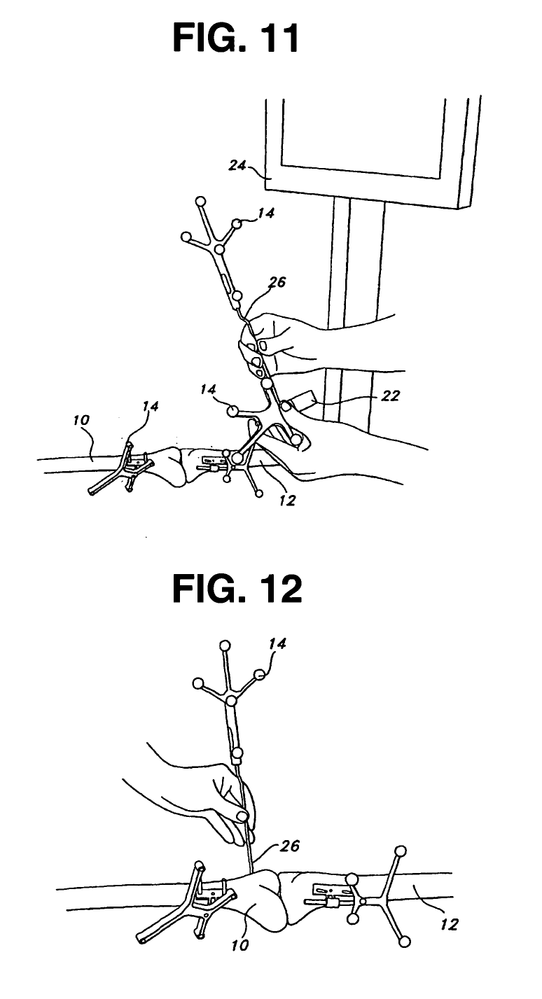

FIG. 11 shows designation or registration of items 22 that will be used in surgery. Registration simply means, however it is accomplished, ensuring that the computer knows which body part, item or construct corresponds to which fiducial or fiducials, and how the position and orientation of the body part, item or construct is related to the position and orientation of its corresponding fiducial or a fiducial attached to an impactor or other other component that is in turn attached to an item. Such registration or designation can be done before or after registering bone or body parts as discussed with respect to FIGS. 4-10. FIG. 11 shows a technician designating with probe 26 an item 22, such as an instrument component, to which fiducial 14 is attached. The sensor 16 “sees” the position and orientation of the fiducial 14 attached to the item 22 and also the position and orientation of the fiducial 14 attached to the probe 26 whose tip is touching a landmark on the item 22. The technician designates onscreen or otherwise the identification of the item 22 and then activates the foot pedal or otherwise instructs the computer 18 to correlate the data corresponding to such identification, such as data needed to represent a particular cutting jig, with the particularly shaped fiducial 14 attached to the cutting jig. The computer 18 has then stored identification, position and orientation information relating to the fiducial for item 22 correlated with the data, such as configuration and shape data, for the item 22 so that upon registration, when sensor 16 tracks the item 22 fiducial 14 in the infrared field, monitor 24 can show the item 22 moving, turning, properly positioned, and oriented relative to the body part that is also being tracked.

### Registration of Anatomy and Constructs

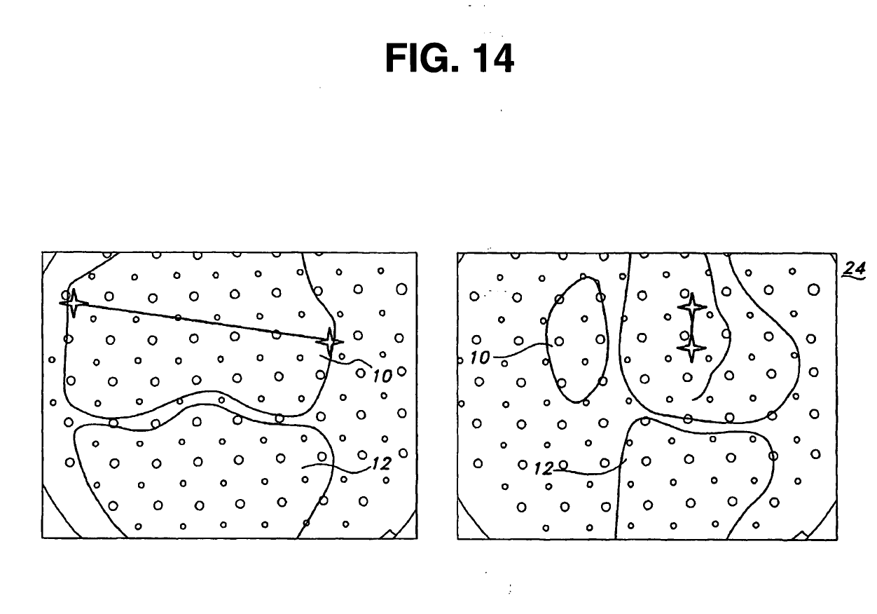

Similarly, the mechanical axis and other axes or constructs of body parts 10 and 12 can also be “registered” for tracking by the system. Again, the system 100 may employ a fluoroscope to obtain images of the femoral head, knee and ankle of the sort shown in FIGS. 4-10. The system 100 correlates such images with the position and orientation of the C-arm and the patient anatomy in real time as discussed above with the use of fiducials 14 placed on the body parts before image acquisition and which remain in position during the surgical procedure. Using these images and/or the probe 26, the surgeon can select and register in the computer 18 the center of the femoral head and ankle in orthogonal views, usually anterior/posterior and lateral, on a touch screen. The surgeon uses the probe 26 to select any desired anatomical landmarks or references at the operative site of the knee or on the skin or surgical draping over the skin; as on the ankle. These points are registered in three-dimensional space by the system 100 and are tracked relative to the fiducials on the patient anatomy which are preferably placed intraoperatively. FIG. 12 shows the surgeon using probe 26 to designate or register landmarks on the condylar portion of femur 10 using probe 26 in order to feed to the computer 18 the position of one point needed to determine, store, and display the epicondylar axis. (See FIG. 14 which shows the epicondylar axis and the anterior-posterior plane and for lateral plane.) Although registering points using actual bone structure, such as in FIG. 12, is one way to establish the axis, a cloud of points approach by which the probe 26 is used to designate multiple points on the surface of the bone structure can be employed, as can moving the body part and tracking movement to establish a center of rotation as discussed above. Once the center of rotation for the femoral head and the condylar component have been registered, the computer 18 is able to calculate, store, and render, and otherwise use data for, the mechanical axis of the femur 10.

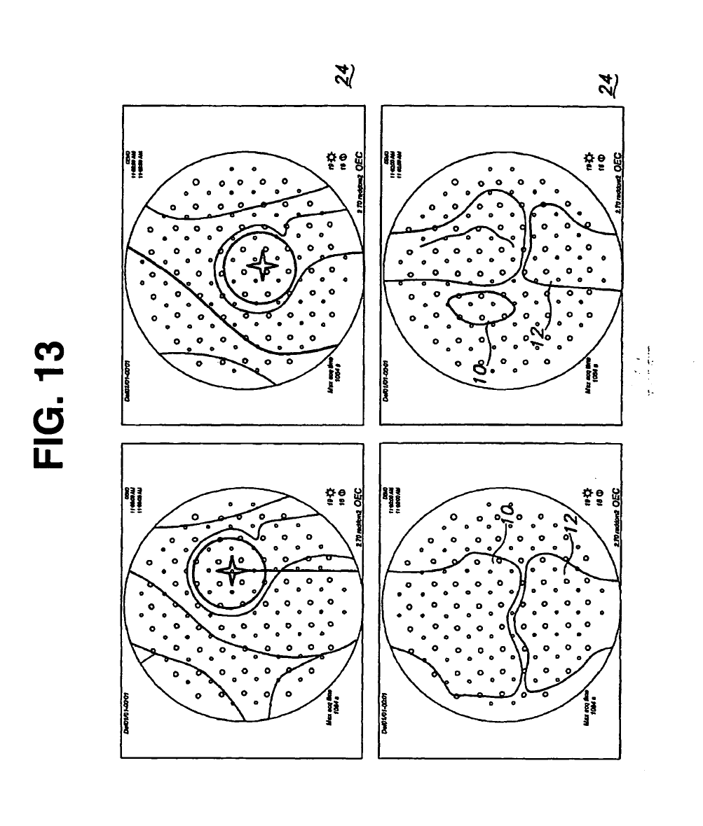

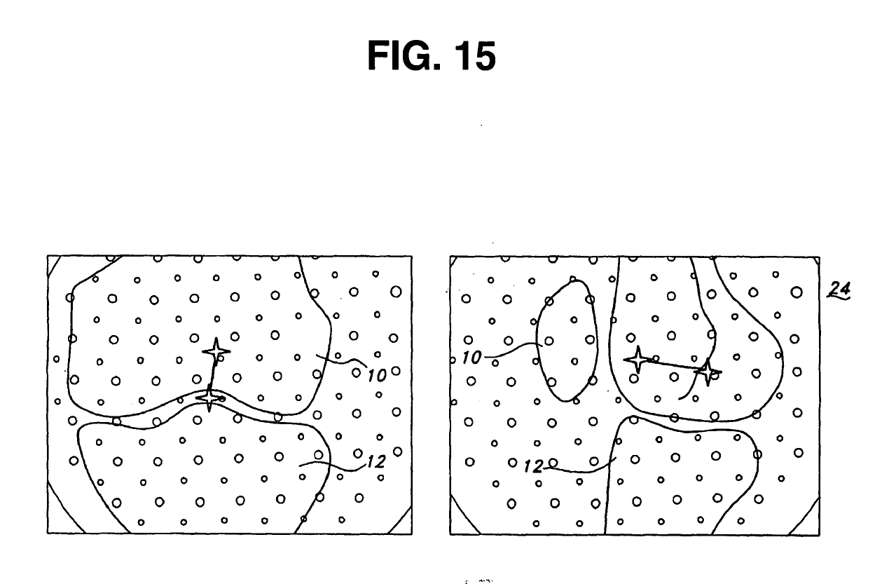

FIG. 13 shows the onscreen images being obtained when the surgeon registers certain points on the bone surface using the probe 26 in order to establish the femoral mechanical axis. The tibial mechanical axis is then established by designating points to determine the centers of the proximal and distal ends of the tibia so that the mechanical axis can be calculated, stored, and subsequently used by the computer 18. FIG. 14 shows designated points for determining the epicondylar axis, both in the anterior/posterior and lateral planes, while FIG. 15 shows such determination of the anterior-posterior axis as rendered onscreen. The posterior condylar axis is also determined by designating points or as otherwise desired, as rendered on the computer generated geometric images overlain or displayed in combination with the fluoroscopic images, all of which are keyed to fiducials 14 being tracked by sensors 16.

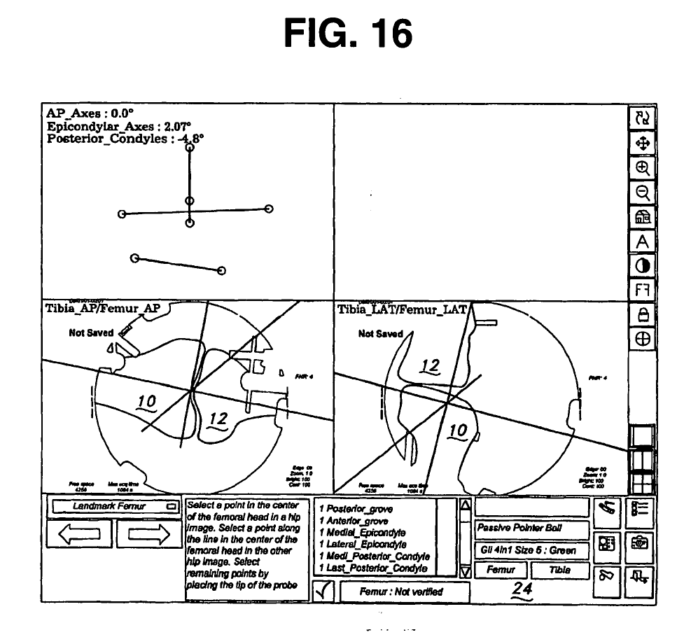

FIG. 16 is an onscreen image showing the anterior-posterior axis, epicondylar axis and posterior condylar axis from points that have been designated as described above. These constructs are generated by the computer 18 and presented on monitor 24 in combination with the fluoroscopic images of the femur 10, correctly positioned and oriented relative thereto as tracked by the system. In the fluoroscopic/computer generated image combination shown at left bottom of FIG. 16, a “sawbones” knee as shown in certain drawings above which contains radio opaque materials is represented fluoroscopically and tracked using sensor 16 while the computer generates and displays the mechanical axis of the femur 10 which runs generally horizontally. The epicondylar axis runs generally vertically, and the anterior/posterior axis runs generally diagonally. The image at bottom right shows similar information in a lateral view. Here, the anterior-posterior axis runs generally horizontally while the epicondylar axis runs generally diagonally, and the mechanical axis generally vertically.

FIG. 16, as is the case with a number of screen presentations generated and presented by the system 100, also shows at center a list of landmarks to be registered in order to generate relevant axes and constructs useful in navigation, positioning and assessment during surgery. Textural cues may also be presented which suggest to the surgeon next steps in the process of registering landmarks and establishing relevant axes. Such instructions may be generated as the computer 18 tracks, from one step to the next, registration of items 22 and bone locations as well as other measures being taken by the surgeon during the surgical operation.

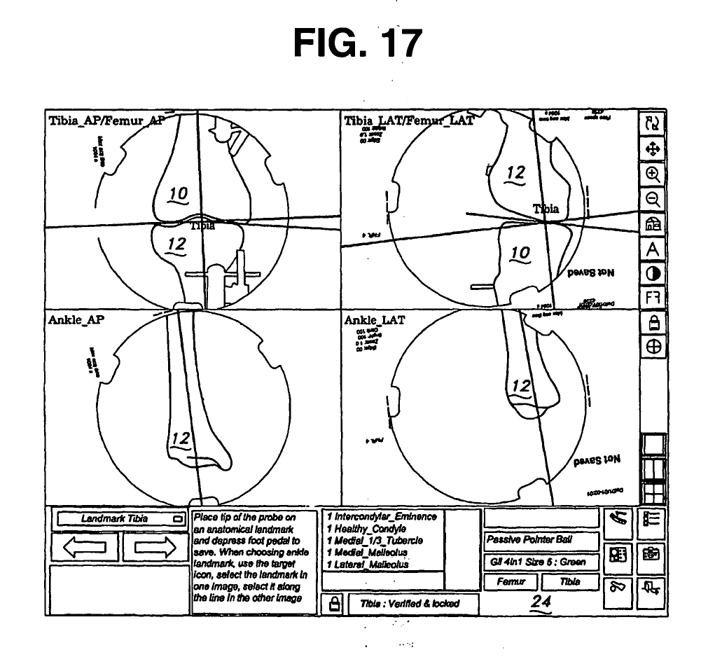

FIG. 17 shows mechanical, lateral, anterior-posterior axes for the tibia according to points are registered by the surgeon.

### Wedge Resection

After the mechanical axis and other rotation axes and constructs relating to the femur and tibia are established, instrumentation can be properly oriented to resect or modify bone in order to properly resect a bone wedge. Instrumentation such as, for instance, cutting jigs, to which fiducials 14 are mounted, can be employed. The system 100 can then track instrumentation as the surgeon manipulates it for optimum positioning. In other words, the surgeon can “navigate” the instrumentation for optimum positioning using the system and the monitor. In this manner, instrumentation may be positioned in order to align the ostetomies to the mechanical and rotational axes or reference axes. The monitor 24 can then also display the instrument such as the cutting jig and/or the pivot pin relative to the cutting jig during this process, in order, among other things, properly to resect a wedge of bone. As the cutting jig moves, the varus/valgus, flexion/extension and internal/external rotation of the relative cutting jig position can be calculated and shown with respect to the referenced axes; in the preferred embodiment, this can be done at a rate of six cycles per second or faster. The cutting jig position is then fixed in the computer and physically, and the surgeon makes the bone wedge resections.

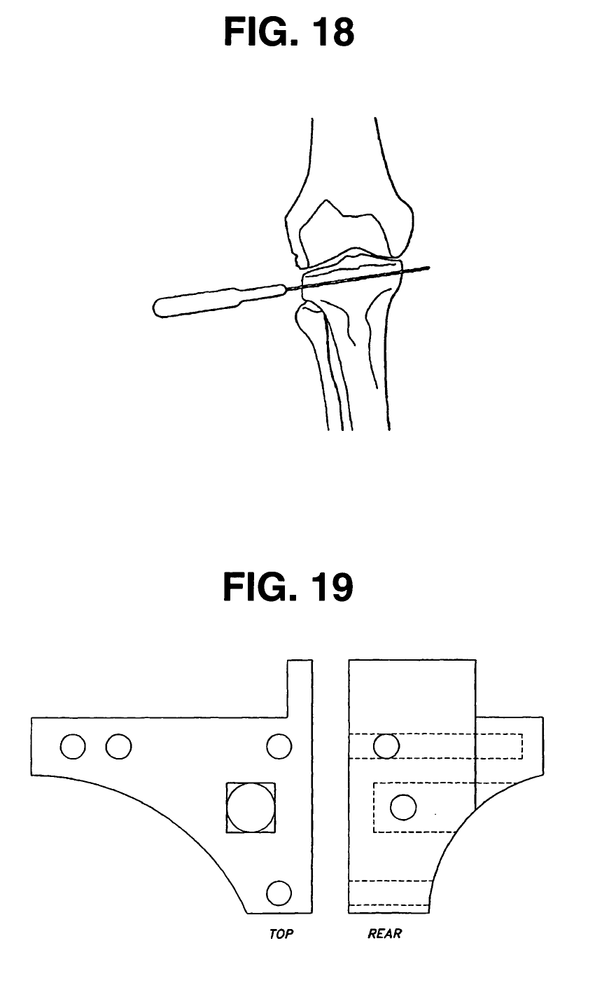

FIG. 18 shows the placement of a pivot pin to which a fiducial is attached via a drill sleeve. The system navigates the placement of a pivot pin at a level of 1 cm from the medial cortex of the tibia and 1 cm below the level of the tibial plateau. The pin is placed perpendicular to the frontal plane and parallel to the sagittal plane. The pivot pin acts as an intersection point for two resection planes of the wedge.

FIG. 19 shows tibial cutting jigs. The system 100 navigates two cutting jigs on an assembly that slides over the pivot pin. The proximal jig is aligned parallel to the tibial plateau and fixed to the tibia, as shown in FIG. 20. The distal jig is then placed radially about the pivot pin.

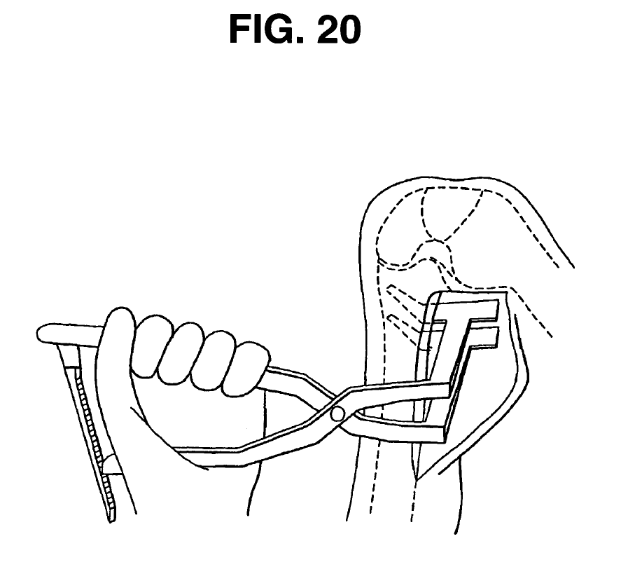

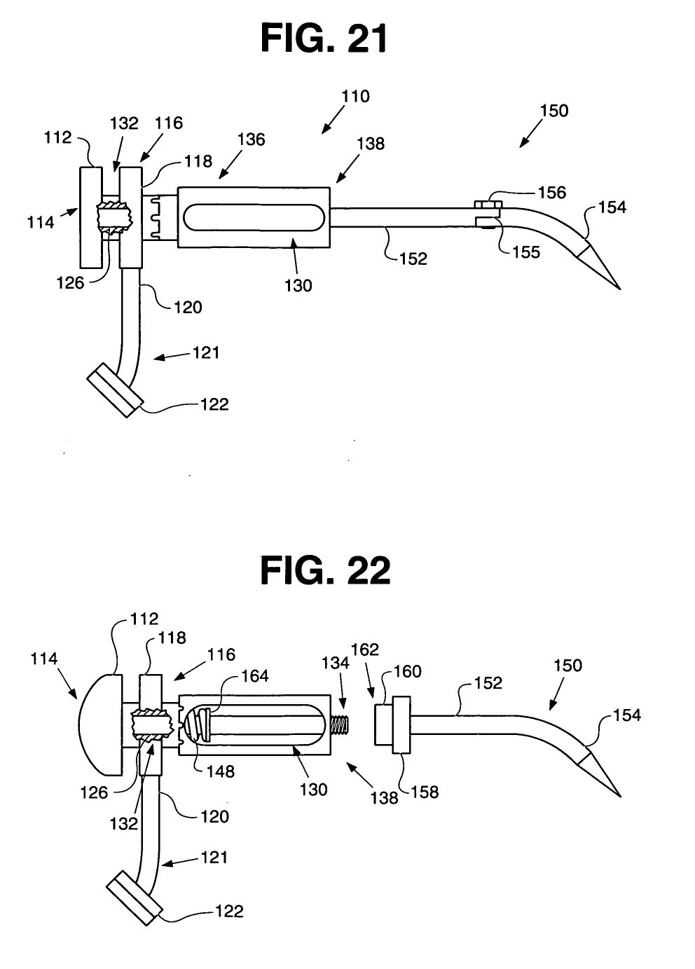

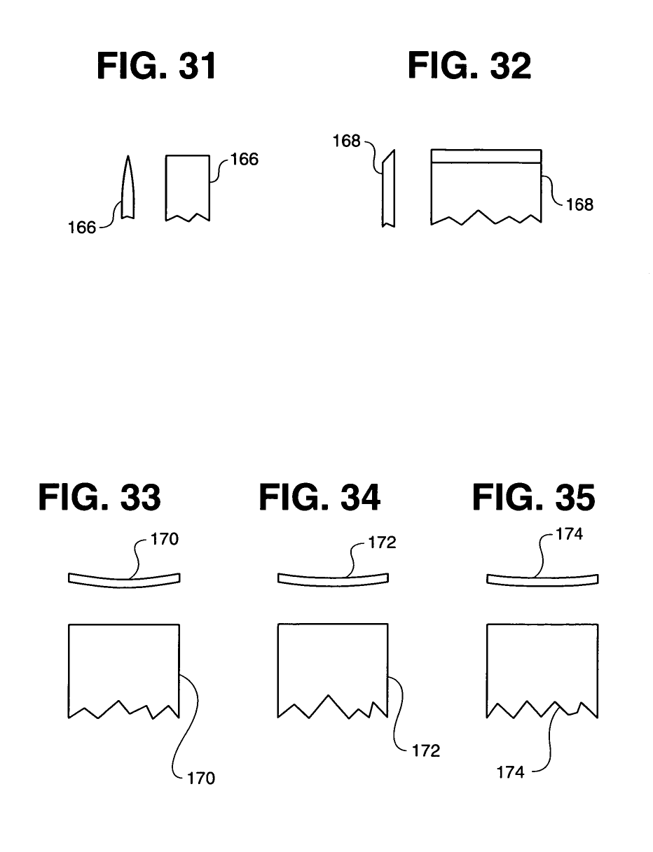

FIG. 21 shows an osteotome instrument 110 for use in computer assisted surgery. For example, the instrument 110 may be used with the system 100 for resecting bone or soft tissue. The instrument 110 includes a connector 116, a handle 124, and a cutter component 150. Optionally, the instrument 110 may also include an impact member 112. Cutter component 150 includes a beam portion 152 and a tip portion 154. Tip portion 154 may include any number of osteotome tip shapes. FIGS. 31-35 illustrate various osteotome tip shapes that may be incorporated into the tip portion 154.

Impact member 112 has an impact surface 114. In general, a surgeon hits the impact surface 114 with a hammer or other tool in order to apply a dynamic force to the instrument 110. Impact surface 114 may have any number of shapes. As examples, the impact surface 114 may be generally planar as depicted in FIG. 21, or, alternatively, the impact surface 114 may be spherical or dome-shaped as depicted in FIG. 22.

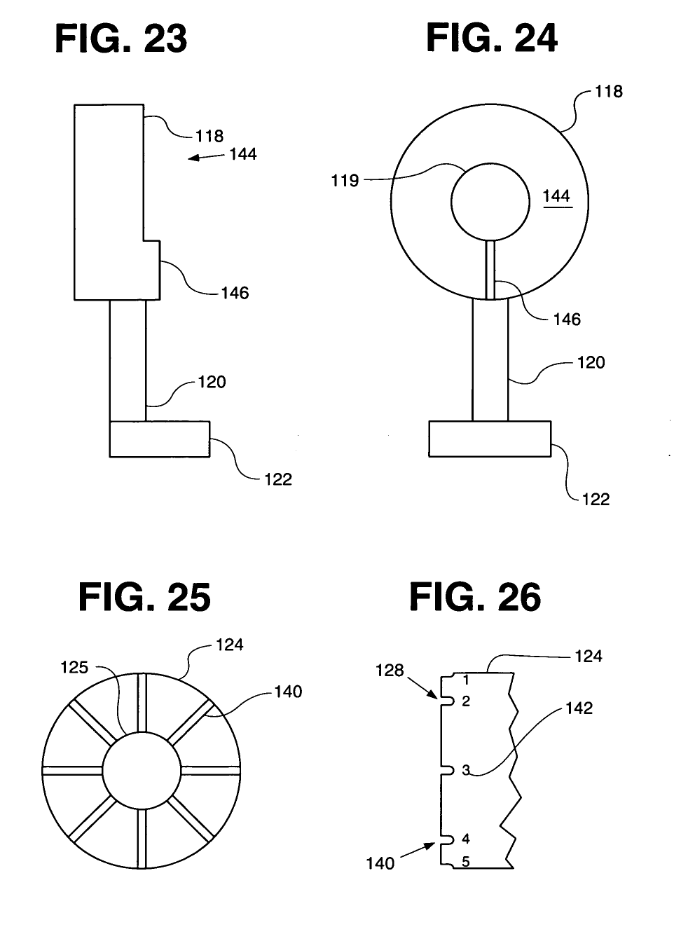

Connector 116 is generally adjacent or juxtaposed to the handle 124. Connector 116 may also be referred to a clocking mechanism or a rotation mechanism. Connector 116 includes a frame 118. In the embodiment depicted in FIGS. 23 and 24, the frame 118 is cylindrical but other shapes may be used. A leg 120 is operatively connected to the frame 118. Leg 120 may be integral with the frame 118 or it may be a separate component. A platform 122 is operatively connected to the leg 120. Platform 122 may be integral with the leg 120 or it may be a separate component. Platform 122 is adapted to receive a fiducial 14. In the embodiment depicted in FIGS. 23 and 24, leg 120 generally extends radially from the frame 118, and the platform 122 is generally perpendicular to the leg 120. In the embodiment depicted in FIGS. 21 and 22, however, leg 120 extends from the frame 118 and includes an arcuate portion 121 such that the platform 122 is angled relative to the main body of the leg 120.

Handle 124 is generally adjacent or juxtaposed to the connector 116. Handle 124 has a proximal portion 136 and a distal portion 138. In some embodiments, handle 124 has one or more openings 130, such as a hole, slot or groove. Connector 116 is located at or near the proximal portion 136, and the cutter component 150 is located on or at the distal portion 138. Connector 116 is adapted to rotate relative to the handle 124. In some embodiments, the instrument 110 includes a locking mechanism to temporarily hold the connector 116 in a position relative to the handle 124. For example, the handle 124 may have a keyway and the connector 116 may have a corresponding key, or vice versa, to hold the connector 116 in a position relative to the handle 124. This example is depicted in FIGS. 23-26.

FIGS. 23-26 illustrate one embodiment of the connector 116 and the handle 124. The connector 116 includes the frame 118, the leg 120, and the platform 122. Connector 116 also includes a face 144 and a key 146. Handle 124 includes an end face 128 and a keyway 140. End face 128 is adapted to mate with the face 144, and the key 146 is adapted to mate with the keyway 140. Although FIGS. 23-26 illustrate the connector 116 as having a key and the handle 124 as having a keyway, those of ordinary skill in the art would understand that the location of such features could be reversed. The handle 124 may have one or more keyways 140. In the embodiment depicted in FIG. 25, the handle 124 has eight keyways 140 but a greater or lesser number of keyways may be used. In some embodiments, the handle 124 or the connector 116 include indicia 142 that indicate which particular keyway has been selected. As examples, the indicia 142 may be letters or numbers. The indicia 142 indicate an orientation of the fiducial 14 mounted on the platform 122 relative to the cutter component 150. In this manner, computer 18 can identify a particular orientation of the instrument 110 when a given position is provided. Further, computer 18 may include software which prompts the user to input the corresponding position of the connector 116. Thereafter, the computer 18 may update a file of the instrument 110 and/or may display on the monitor 24 an accurate rendering of the instrument 110. Alternatively, computer 18 may maintain a database of a plurality of files for instrument 110 with each file corresponding to a particular rotational position of connector 116. The computer 18 may retrieve a particular file from the database after a user inputs the particular rotational position.

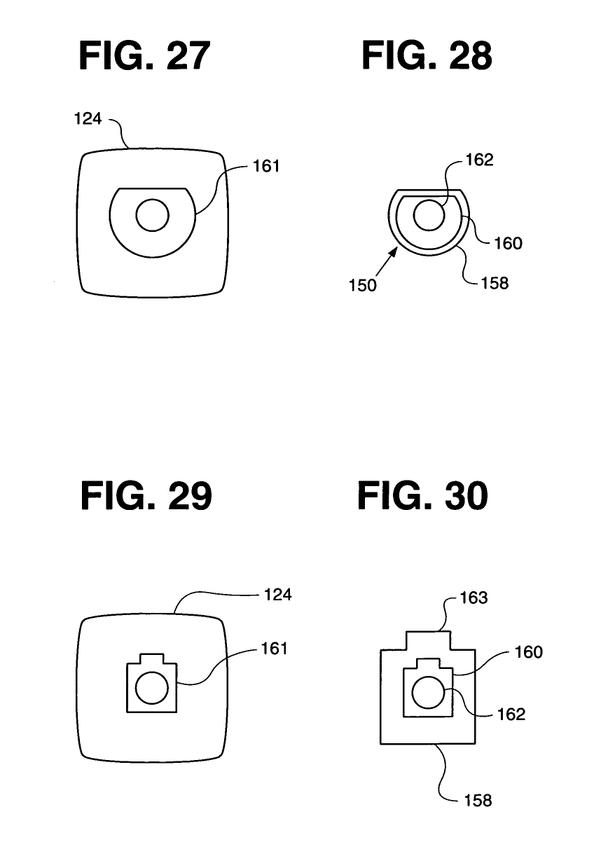

Referring again to FIGS. 21 and 22, the cutter component 150 may be integral with the handle 124 as depicted in FIG. 21, or it may be a separate component as depicted in FIG. 22. If the cutter component 150 is assembled or removably attached to the handle 124 as shown in FIG. 22, the cutter component 150 may have a feature or key that only allows the cutter component to be put on one-way. For example, the cutter component 150 may have a projection 160 and the handle 124 may have a corresponding receiver 161, or vice versa, such that the cutter component 150 can be assembled to handle 124 in one direction. In the embodiment depicted in FIG. 22, the cutter component 150 has a plug 158. The projection 160 and the beam portion 152 each extend from the plug 158, although on different sides. Projection 160 and receiver 161 may have any number of various shapes to achieve the desired function. Two examples of these various shapes are depicted in FIGS. 27-30. In FIGS. 27-28, projection 160 and the receiver 161 have a D-shape. However, in FIGS. 29-30, projection 160 is square-shaped with a tab 163 and receiver 161 has a corresponding shape. The projection 160 is inserted into the receiver 161 until the distal portion 138 of the handle 124 substantially contacts the plug 158.

Referring once again to FIGS. 21 and 22, the tip portion 154 of the cutter component 150 may be integral with the beam portion 152 as depicted in FIG. 22 or the tip portion 154 may be a separate component as depicted in FIG. 21. A separate component would allow the tip portion 154 to be replaced if it becomes bent, dull, or broken. In the case of a separate component, the tip portion 154 and the beam portion 152 may each include a notch 155, or some other locating feature, to align and locate the components relative to one another. In some embodiments, cutter component 150 includes a fastener 156, such as a bolt or a screw, in order to secure the tip portion 154 to the beam portion 152.

Instrument 110 also includes a shaft 126. Impact member 112, connector 116, and the handle 124 are assembled about the shaft 126. The shaft 126 has a first end portion 132 and a second end portion 134. Impact member 112 is operatively connected to the shaft 126 at the first end portion 132. As an example, the shaft 126 may threadingly engage the impact member 112. The connector 116 includes an aperture 119, and the shaft 126 is inserted through the aperture 119. Handle 124 includes a cutout 125. In the embodiment depicted in FIG. 25, the cutout 125 is cylindrical but other shapes may be used. In embodiments where the handle 124 and the cutter component 150 are integrally formed together, the shaft 126 is operatively connected to the handle 124. For example, the shaft 126 may threadingly engage the handle 124. In other embodiments, however, the shaft 126 extends through the handle 124 and is operatively connected to the cutter component 150 at the second end portion 134. For example, the cutter component 150 may have a threaded hole 162, and the shaft 126 threadingly engages the hole 162.

Connector 116 rotates about the shaft 126. Because some tip portions 154 are straight, some are angled, and some are curved, the geometry is not axis symmetric. Rotation of connector 116 allows the platform 122 and fiducial 14 to move about the shaft 126 and yet allow computer 18 to understand the orientation of the tip portion 154.

In some embodiments, the connector 116 is biased towards the handle 124. This allows the temporary locking mechanism to positively engage in order to keep the connector 116 in a selected position. In the embodiment depicted in FIG. 22, the instrument 110 includes a retaining ring or clip 164 operatively connected to the shaft 126. For example, the clip 164 may positively engage the shaft 126 for temporary fixation or the clip 164 may permanently engage the shaft 126, such as by welding. The instrument 110 also includes a spring 148. The spring 148 slides over the shaft 126 and engages both an interior portion (not shown) of the handle 124 and the clip 164. The spring 148 pushes on the clip 164. Because the clip 164 is operatively connected to the shaft 126, the shaft 126 pulls on the impact member 112, which biases the connector 116 toward the handle 124. A user may pull on the connector 116 with sufficient force to overcome the spring 148 in order to rotate the connector 116. This would allow the leg 120, and thus the fiducial 14, to be moved relative to the handle 124 and/or the cutter component 150. When the instrument 110 includes a locking mechanism, such as key 146 and keyway 140, the connector 116 is temporarily fixed in a position relative to the handle 124.

As noted above, FIGS. 31-35 illustrate various osteotome tip shapes that may be incorporated into the tip portion 154. The tip portion 154 may have a blade tip 166, a chisel tip 168, a 50 mm radius gouge 170, a 60 mm radius gouge 172, or a 70 mm radius gouge 174.

In order to use the instrument 110 with the system 100, it is generally best to calibrate the tip portion 154. In other words, it is important for the system 100 to understand the position and orientation of the tip portion 154 so that the surgeon can reliably and accurately carry out the procedure. Thus, a calibration unit is needed to calibrate the tip portion 154.

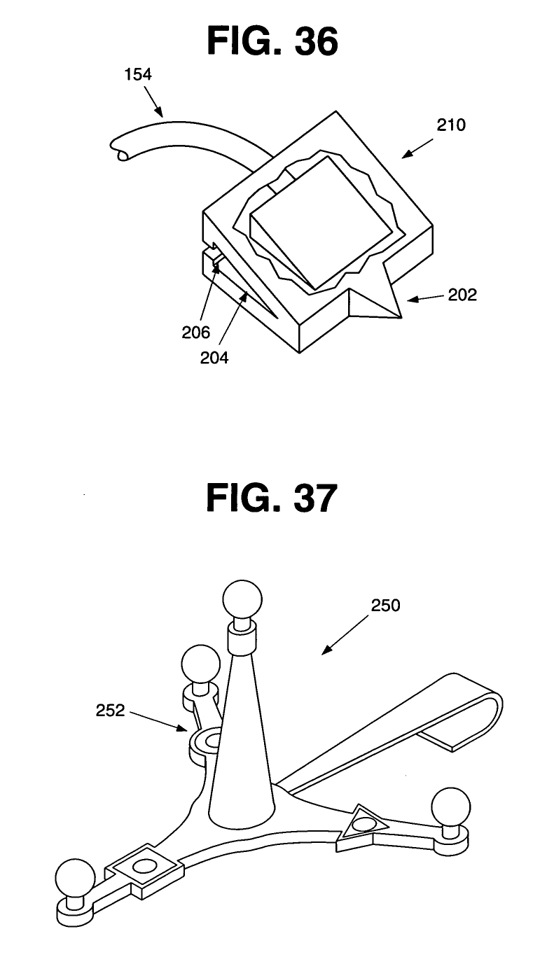

FIG. 36 illustrates a first embodiment of the calibration unit, generally indicated by reference numeral 210. The calibration unit 210 includes a verification point 202 and an interior pocket 204. In the embodiment depicted in FIG. 36, the calibration unit 210 is a clasp or receptacle that fits over and snaps onto the tip portion 154. The interior pocket 204 is sized and dimensioned to fully capture the tip portion 154 and eliminate or substantially reduce side-to-side motion of the calibration unit 210. In some embodiments, a lip 206 of the interior pocket 204 engages a back edge of the tip portion 154. Verification point 202 is adapted for use with a reference frame divot 252 of a fiducial 250 (best seen in FIG. 37). Calibration unit 210 is made from a material that is semi-rigid to allow the receptacle 210 to slide over the tip portion and positively lock in position. In some embodiments, the verification point 202 is rigid to reduce wear and increase repeatability of calibration.

In operation, a user places the receptacle 210 over the tip portion 154 and places the verification point 202 in a reference frame divot 252 of a fiducial 250. Thereafter, the user indicates to the computer 18 that the tip portion is ready for calibration. As examples, this may be accomplished by touching the screen 24, depressing the foot pedal 20, or holding the instrument 110 steady for a period of time. Thereafter, the computer 18 tracks the fiducial 14 mounted to the platform 122 and “memorizes” the orientation and position of the tip portion 154.

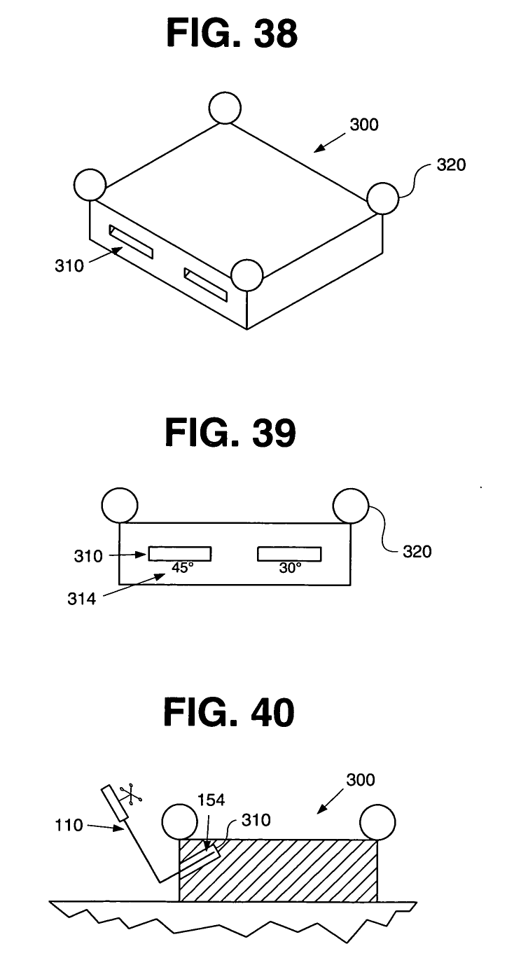

FIGS. 38-40 illustrate another embodiment of the calibration unit, generally indicated by reference numeral 300. In the embodiment depicted in FIGS. 38-40, the calibration unit is a calibration block having a slot 310. Although the calibration unit 300 is shown with two slots in FIGS. 38-40, those skilled in the art would understand the calibration block may have any number of slots depending upon the size of the block and the size of the tip portion 154. Slot 310 allows computer 18 to determine array position with respect to the tip portion 154. The slots 310 may be angled to accommodate an angled tip portion 154. For example, the slots 310 may have an angle of about 35 degrees or about 45 degrees. Further, in some embodiments, slot 310 may be angled upwardly such that tip portion 154 may be inserted only one-way and prevent the handle 124 from hitting a surface below the calibration block 300, such as the top of a table. Each slot 310 is sized and dimensioned to receive the tip portion 154. Further, calibration unit 300 may include characters 314 to indicate the angle of the particular slot.

Calibration unit 300 further includes reflective elements or spheres 320, which may be tracked by sensor 16. The spheres 320 may be active or passive. In the embodiment depicted in FIGS. 38-40, the calibration unit 300 has four spheres but a greater or lesser number of spheres may be used. Although reflective elements 320 are depicted as having a spherical shape, those of ordinary skill in the art would understand that reflective elements 320 may have any number of different shapes. What is significant is that reflective elements 320 can be tracked by position orientation sensor 16.

System 100 verifies or calculates the position and/or orientation of the tip portion 154 by comparing the position of the spheres 320 and the array 14 mounted to the platform 122 and the known position of the slot with the calculated position of the array 14. Calibration block 300 allows placement of the array anywhere on the instrument because verification of the tip portion 154 occurs via spheres 320 and the relative position of the array. Thus, the array may be rotated to a position for optimal comfort and/or optimal visibility by sensor 16.

In operation and with reference to FIG. 40, the tip portion 154 of the instrument 110 is inserted into a slot 310. Thereafter, the user indicates to the computer 18 that the tip portion is ready for calibration. As examples, this may be accomplished by touching the screen 24, depressing the foot pedal 20, or holding the instrument 110 steady for a period of time. Thereafter, the computer 18 tracks the fiducial 14 mounted to the platform 122 and “memorizes” the orientation and position of the tip portion 154.

It may also be important for the system 100 to identify the configuration of the cutter component 150. For example, the tip portion 154 may be thin, thick, curved or straight. For this purpose, instrument 110 may employ unique navigation array geometry or other identifier to indicate to the computer 18 the shape of the tip portion 154. Further, a geometrically appropriate calibration block may be employed so that computer 18 may calculate the configuration and orientation of tip portion 154. For example, the calibration block may have slots of certain widths, slots adapted only to receive a straight blade, or slots adapted only to receive a curved blade. Finally, it may be possible to digitize a few key points of the tip portion 154 to indicate to computer 18 the particular configuration.

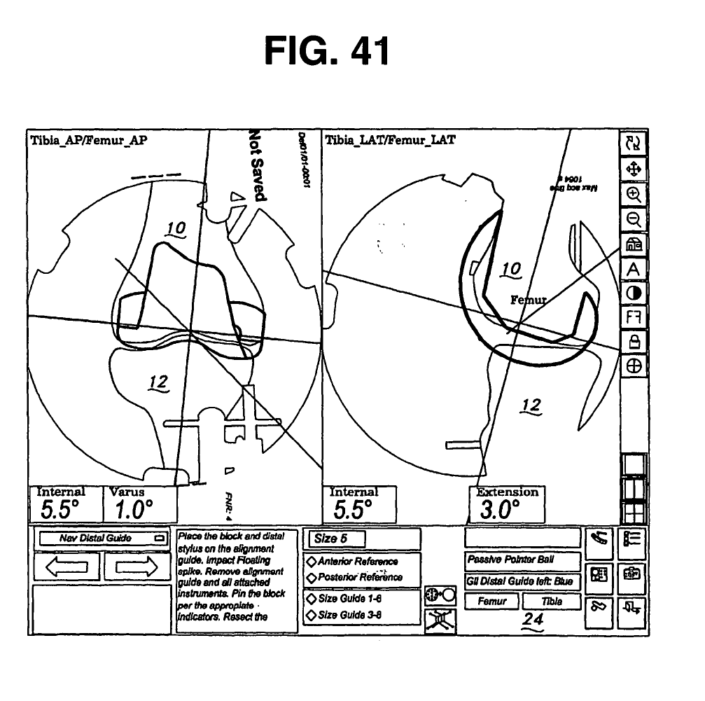

FIG. 41 also shows other information relevant to the surgeon such as the name of the component being overlain on the tibial image, suggestions or instructions at the lower left, and angle of the rod in varus/valgus and extension relative to the axes. Any or all of this information can be used to navigate and position the cutting jig relative to the tibia.

### Navigation, Placement and Assessment of Angle

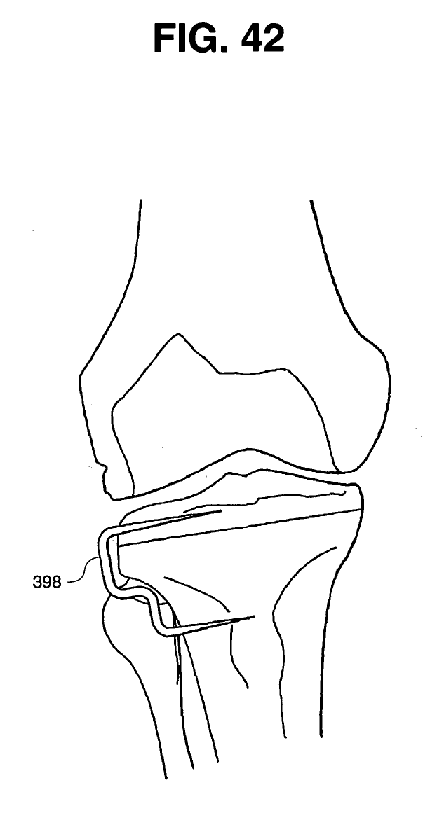

Once the distal jig is placed radially about the pivot pin, the jig is adjusted radially to the desired angle calculated by the system 100 based on desired correction algorithms and reference axes. The distal jig is fixed to the tibia and the bone wedge is resected. After removal of the wedge, either the opening is reduced and plated or stapled 398 for a closed wedge procedure, as shown in FIG. 42, or it is braced open with a plate for an open wedge procedure. The open wedge is then grafted to fill the void.

During the wedge resection process, instrument positioning process or at any other desired point in surgical or other operations, the system 100 can transition or segue from tracking a component according to a first fiducial to tracking the component according to a second fiducial. Thus, the pivot pin can be mounted on a drill sleeve to which a fiducial 14 is attached. The pivot pin is installed and positioned using the drill sleeve. The computer 18 “knows” the position and orientation of the pin relative to the fiducial on the drill sleeve (such as by prior registration of the component attached to the drill sleeve) so that it can generate and display the image of the pivot pin on screen 24 overlaid on the fluoroscopic image of the tibia. At any desired point in time, before, during or after the pivot pin is properly placed in the tibia to align with mechanical axis and according to proper orientation relative to other axes, the system 100 can be instructed by foot pedal or otherwise to begin tracking the position of the pivot pin using the fiducial attached to the tibia rather than the one attached to the drill sleeve. In some embodiments, the sensor 16 “sees” at this point in time both the fiducials on the drill sleeve and the tibia 12 so that it already “knows” the position and orientation of the pivot pin relative to the fiducial on the drill sleeve and is thus able to calculate and store for later use the position and orientation of the pivot pin relative to the tibia 12 fiducial. Once this “handoff” happens, the drill sleeve can be removed and the pivot pin tracked with the tibia fiducial 14 as part of or moving in concert with the tibia 12. Similar handoff procedures may be used in any other instance as desired.

U.S. Patent Application Publication No. 2005/0234332A, published on Oct. 20, 2005, having an Application No. of Ser. No. 11/037,898, filed on Jan. 18, 2005, and listing Stephen B. Murphy as the sole inventor, the disclosure of which is incorporated by reference herein, discloses systems, methods, and processes for computer-assisted soft tissue balancing, including ligament balancing. Instrument 110 is well-suited for use in such soft-tissue balancing.

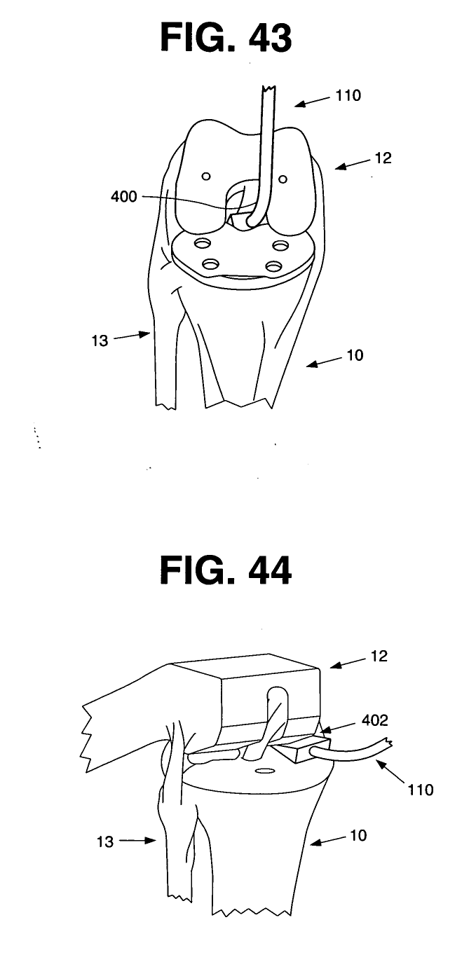

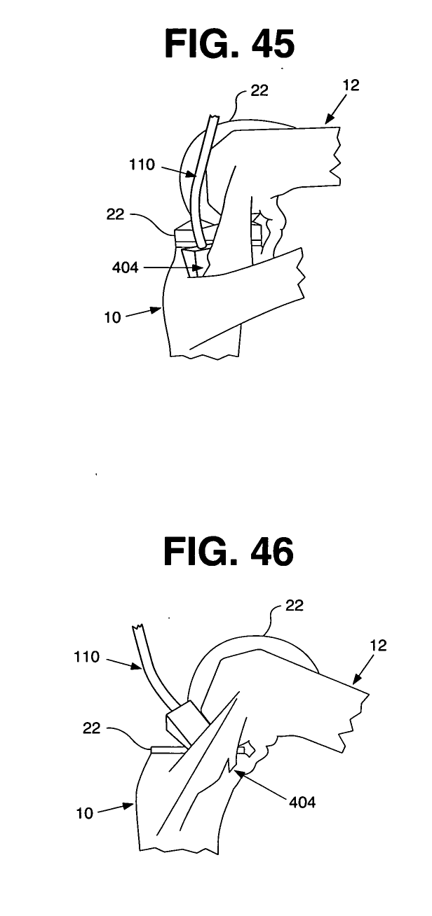

FIGS. 43-52 illustrate use of the osteotome instrument in soft tissue balancing. In FIG. 43, a surgeon utilizes the instrument 110 to release the posterior cruciate ligament 400. The tip portion 154 is used to make several small cuts around the posterior cortical margin in order to loosen a small segment of bone from its posterior tibial attachment. Also depicted in FIG. 43 are the tibia 10, the femur 12, and the fibula 13. A surgeon uses the instrument 110 having a curved tip portion to cut free the posterior femoral osteophytes 402 in FIG. 44. In FIG. 45, a surgeon releases the anterior fibers 404 utilizing the instrument 110. Also depicted in FIG. 45 are items 22, which may be trials or implants.

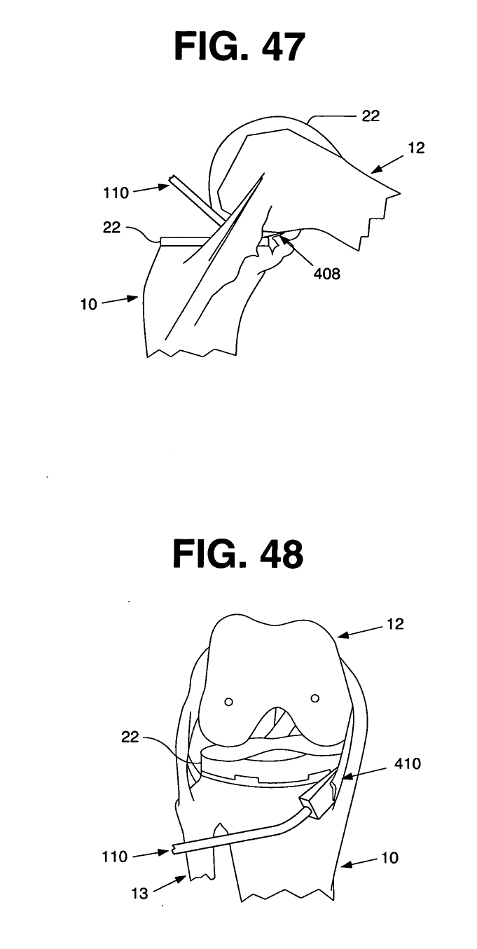

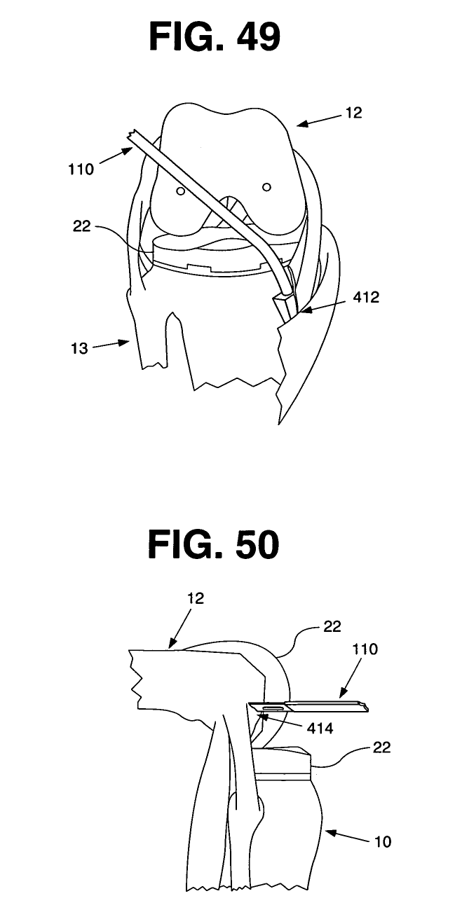

FIG. 46 illustrates the instrument 110 being used to release the medial collateral ligament 406. In FIG. 47, a surgeon utilizes the instrument 110 to release the medial posterior capsule 408. As an example, this may be done if the knee is too tight medially in extension. A surgeon utilizes the instrument 110 to release the medial collateral ligament 406 in FIG. 48. The surgeon inserts the tip portion 154 at an upper, anterior edge 410 of the medial collateral ligament 406. For example, this may be done if the knee is too tight medially in flexion and extension. As best seen in FIG. 49, the tip portion is inserted behind the pes anserinus 412 to strip subperiosteally the medial collateral ligament.

FIGS. 50 and 51 illustrate an instrument 110 having a tip portion 154 in the shape of a scalpel blade. In the embodiment depicted in FIG. 50, the instrument 110 is used to release the popliteus tendon 414 from the femur 10. Similarly, FIG. 51 illustrates the instrument 110 being used to release the iliotibial band 416. This may be done if the knee joint remains tight laterally in extension.

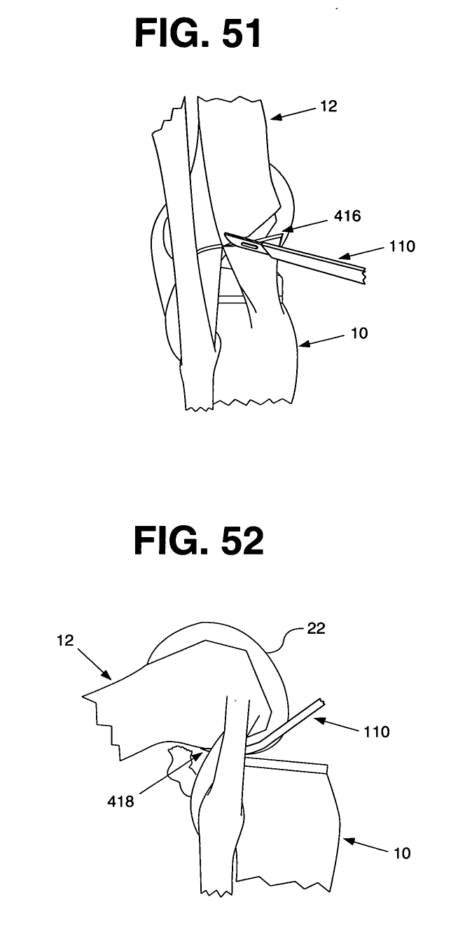

In the embodiment depicted in FIG. 52, a surgeon uses instrument 110 to release the lateral posterior capsule. As an example, this technique may be applied in the rare case when the knee exhibits lateral tightness in full extension after release of the iliotibial band 416.

At the end of the case, all alignment and/or balancing information can be saved for the patient file. This is of great assistance to the surgeon due to the fact that the outcome of implant positioning can be seen before any resectioning has been done on the bone. The system 100 is also capable of tracking the patella and resulting placement of cutting guides and the patellar trial position. The system 100 then tracks alignment of the patella with the patellar femoral groove and will give feedback on issues, such as, patellar tilt.

The tracking and image information provided by the system 100 facilitate telemedical techniques, because they provide useful images for distribution to distant geographic locations where expert surgical or medical specialists may collaborate during surgery. Thus, the system 100 can be used in connection with computing functionality 18 which is networked or otherwise in communication with computing functionality in other locations, whether by public switched telephone network (PSTN), information exchange infrastructures, such as packet switched networks including the Internet, or as otherwise desire. Such remote imaging may occur on computers, wireless devices, videoconferencing devices or in any other mode or on any other platform which is now or may in the future be capable of rending images or parts of them produced in accordance with the present invention. Parallel communication links, such as switched or unswitched telephone call connections may also accompany or form part of such telemedical techniques. Distant databases, such as online catalogs of implant suppliers or prosthetics buyers or distributors, may form part of or be networked with functionality 18 to give the surgeon in real time access to additional options for implants which could be procured and used during the surgical operation.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An instrument comprising:
a. a shaft;
b. a handle having a proximal end portion and a distal end portion;
c. a cutter component operatively connected to said handle at said distal end portion;
d. a connector releasably connected to said handle at said proximal end portion, said connector adapted to rotate about said shaft relative to said handle; and
e. a fiducial operatively connected to said connector.

2. The instrument according to claim 1, further comprising a spring adapted to bias said connector toward said handle and a retaining ring operatively connected to said shaft.

3. The instrument according to claim 1, wherein said handle is operatively connected to said shaft.

4. The instrument according to claim 1, further comprising an impact member operatively connected to said shaft.

5. The instrument according to claim 1, wherein said cutter component further comprises a beam and a tip portion operatively connected to said beam.

6. The instrument according to claim 5, wherein said tip portion is selected from the group consisting of a blade, a chisel, a gouge, and a scalpel.

7. An osteotome instrument for use in computer assisted surgery, the instrument comprising:
a. a shaft having a first end portion and a second end portion;
b. an impact member operatively connected to said shaft and located at said first end portion;
c. a connector slidably connected to said shaft;
d. a fiducial operatively connected to said connector;
e. a handle juxtaposed said connector and mounted about said shaft, said handle having a proximal portion and a distal portion; and
f. a cutter component operatively connected to said handle at said distal portion.

8. The instrument according to claim 7, wherein said cutter component is removably attached to said handle.

9. The instrument according to claim 7, wherein an end face of said handle engages a face of said connector.

10. The instrument according to claim 7, wherein said cutter component is operatively connected to said shaft at said second end portion.

11. The instrument according to claim 7, wherein said handle and said cutter component are keyed so that a portion of said cutter component can be inserted into a portion of said handle in only one configuration.

12. The instrument according to claim 7, further comprising a spring adapted to bias said connector toward said handle and a retaining ring operatively connected to said shaft.

13. The instrument according to claim 7, wherein said connector includes indicia that indicate an orientation of said fiducial relative to said cutter component.

14. The instrument according to claim 7, wherein said cutter component threadingly engages said shaft.

15. The instrument according to claim 7, wherein said impact member has a domed surface.

16. The instrument according to claim 7, wherein said handle is operatively connected to said shaft.

17. The instrument according to claim 7, wherein said handle further comprises a cutout and said shaft is inserted through said cutout.

18. The instrument according to claim 7, wherein said handle is slidably connected to said shaft.

19. The instrument according to claim 7, wherein said handle further comprises at least one opening.

20. The instrument according to claim 7, wherein said connector has a cylindrical frame.

21. The instrument according to claim 7, wherein said handle includes at least one keyway and said connector has at least one key adapted to mate with said keyway.

22. The instrument according to claim 7, wherein said connector includes at least one keyway and said handle has at least one key adapted to mate with said keyway.

23. The instrument according to claim 7, wherein said connector further comprises a leg and a platform, and said fiducial is connected to said platform.

24. The instrument according to claim 23, wherein said leg further comprises an arcuate portion.

25. The instrument according to claim 7, wherein said handle further comprises a receiver and said cutter component further comprises a projection adapted to mate with said receiver.

26. The instrument according to claim 25, wherein said projection is D-shaped.

27. The instrument according to claim 7, wherein said cutter component further comprises a beam and a tip portion operatively connected to said beam.

28. The instrument according to claim 27, wherein said tip portion is selected from the group consisting of a blade, a chisel, and a gouge.

29. The instrument according to claim 27, wherein said tip portion is integral with said beam.

30. The instrument according to claim 27, wherein said tip portion is removably attached to said beam.

31. The instrument according to claim 27, wherein said tip portion and said beam portion each include a notch.

32. A system for performing computer assisted surgery, the system comprising:
a. a first fiducial operatively connected to a first part;
b. a second fiducial operatively connected to a second part;
c. at least one position and orientation sensor adapted to track said first fiducial and said second fiducial;
d. a computer having a memory, a processor, and an input/output device, said input/output device adapted to receive data from said at least one position and orientation sensor relating to a position and an orientation of said first fiducial and said second fiducial;
e. a monitor operatively connected to said input/output device of said computer;
f. an osteotome instrument, said instrument comprising:
i. a shaft;
ii. a handle having a proximal end portion and a distal end portion;
iii. a cutter component operatively connected to said handle at said distal end portion;
iv. a connector releasably connected to said handle at said proximal end portion, said connector adapted to rotate about said shaft relative to said handle; and
v. a fiducial operatively connected to said connector; and
g. a calibration unit, said calibration unit adapted to receive at least a portion of said cutter component of said osteotome instrument.

33. The system according to claim 32, wherein said calibration unit is a receptacle.

34. The system according to claim 32, wherein said calibration unit is a calibration block.
